(19) United States
(12) Patent Application Publication
(10) Pub. No.: US 2026/0041706 A1
(43) Pub. Date: Feb. 12, 2026

(54) MUCUS MEMBRANE FORMULATIONS AND USES THEREOF

(71) Applicant: The Brigham and Women's Hospital, Inc., Boston, MA (US)

(72) Inventors: Jeffrey M. KARP, Chestnut Hill, MA (US); Nitin JOSHI, Boston, MA (US); John JOSEPH, Boston, MA (US); Yohannes TESFAIGZI, Brookline, MA (US); Helna Mary BABY, Boston, MA (US)

(21) Appl. No.: 19/100,622

(22) PCT Filed: Aug. 4, 2023
(86) PCT No.: PCT/US2023/029540

§ 371 Date: Feb. 3, 2025

Related U.S. Application Data
(60) Provisional application No. 63/395,088, filed on Aug. 4, 2022.

(51) Int. Cl. A61K 31/732; A61K 9/00; A61K 9/08; A61K 31/045; A61K 31/14; A61K 31/4045; A61K 31/485; A61K 31/717; A61K 31/723; A61K 31/731; A61K 31/765; A61K 45/06; A61P 31/04; A61P 31/14; A61P 31/16; A61P 39/00

(52) U.S. Cl. CPC A61K 31/732; A61K 9/006; A61K 9/08; A61K 31/045; A61K 31/14; A61K 31/4045; A61K 31/485; A61K 31/717; A61K 31/723; A61K 31/731; A61K 31/765; A61K 45/06; A61P 31/04; A61P 31/14; A61P 31/16; A61P 39/00

(57) ABSTRACT

The invention features a mucosal coating composition including: (i) water: (ii) at least one mucoadhesive polymer/polysaccharide (e.g., a concentration between 0.1-20% w/v); and (iii) at least one surfactant (e.g., a concentration between 0.005-5% w/v). Exhibit long-lasting residence times on mucosal tissues and prophylactic protection against transmucosal pathogens.

### BACKGROUND OF THE INVENTION

The past few decades have witnessed numerous instances of disease outbreaks due to infections of mucus membrane such as those caused by respiratory pathogens among others, often leading to epidemics or unanticipated pandemics such as COVID-19. These pathogens, for example, are a prevalent cause of upper and lower respiratory tract infections. One of the predominant modes of pathogen transmission is through inhalation of contaminated respiratory droplets and their subsequent deposition in the nasal cavity. Most respiratory viruses have an entry checkpoint in the nasal cavity due to tissue tropism and receptors. For instance, the SARS-CoV-2 virus binds to the angiotensin-converting enzyme receptors located in goblet cells via its receptor-binding domain (RBD) and continues its replication cycle, spreading infection in the airways. A need in the art accordingly exists for nasal formulations to combat respiratory pathogens.

### SUMMARY OF THE INVENTION

The invention features a mucosal coating composition including: (i) water; (ii) at least one mucoadhesive polymer/polysaccharide; and (iii) at least one surfactant.

The invention further features a mucosal coating composition including: (i) water; (ii) at least one mucoadhesive polymer/polysaccharide at a concentration between 0.1-20% w/v (e.g., a concentration of 1±0.5, 3±2, 7±2.5, 10±2.5, 15±2.5, or 17.5±2.5% w/v); and (iii) at least one surfactant at a concentration between 0.005-5% w/v (e.g., a concentration of 0.1±0.05, 0.25±0.15, 0.5±0.25, 1±0.25, 1.5±0.5, 2±1, or 3±2% w/v).

In certain embodiments of the above compositions, following application to a surface the coating composition forms a barrier that reduces small molecule transport by at least 90% (e.g., by at least 95%, 99%, or 99.9%) over a period of 4 hours.

In particular embodiments of the above compositions, following application to a surface the coating composition forms a barrier that reduces virus transport by at least 90% (e.g., by at least 95%, 99%, or 99.9%) over a period of 4 hours.

In certain embodiments of the above compositions, following application to a surface the coating composition increases the capture of nebulized particles by at least 1.5-fold (e.g., by at least 2-fold, 3-fold, or 5-fold).

In particular embodiments of the above compositions, following application to a surface the coating composition increases the residence time of the coating, resulting in at least 2-fold (e.g., at least 5-fold, 10-fold, 15-fold, or 30-fold) higher amount of coating remaining at 8 hours post-application, compared to a control coating otherwise identical in composition but lacking surfactants.

In some embodiments of the above compositions the mucoadhesive polymer/polysaccharide is present at a concentration between 0.25-20% w/v. In other embodiments of the above compositions the mucoadhesive polymer/polysaccharide is present at a concentration between 0.75-20% w/v.

In some embodiments of the above compositions the surfactant is present at a concentration between 0.01-5% w/v. In other embodiments of the above compositions the surfactant is present at a concentration between 0.05-5% w/v. In certain embodiments of the above compositions the surfactant is present at a concentration between 0.5-5% w/v.

The invention features a mucosal coating composition including: (i) water; (ii) at least one mucoadhesive polymer/polysaccharide at a concentration between 0.1-10% w/v (e.g., a concentration of 0.5±0.25, 3±2, 5±2.5, or 7.5±2.5% w/v); and (iii) at least one surfactant at a concentration between 0.005-5% w/v (e.g., a concentration of 0.1±0.05, 0.25±0.15, 0.5±0.25, 1±0.25, 1.5±0.5, 2±1, or 3±2% w/v), wherein the composition is a sprayable solution.

In any of the above compositions, the at least one mucoadhesive polymer/polysaccharide can include a carboxyl, hydroxyl, sulfate or acetamido group. For example, the at least one mucoadhesive polymer/polysaccharide can be selected from gellan, pectin, HPMC, CMC, xanthan, chondroitin sulfate, alginic acid, hyaluronic acid, and salts or derivatives thereof. In one particular embodiment, the at least one mucoadhesive polymer/polysaccharide includes a polymer of glucuronic acid or galacturonic acid (e.g., pectin, gellan, HPMC, CMC, xanthan and alginic acid, and salts or derivatives thereof).

In any of the above compositions, the at least one surfactant can be a (a) hydrophilic and (b) non-ionic or cationic surfactant. For example, the at least one surfactant can be selected from polysorbate surfactants, sorbitan fatty acid ester surfactants, and benzalkonium chloride. The at least one surfactant can be selected from polyoxyethylene 20 sorbitan monolaurate, polyoxyethylene 20 sorbitan monopalmitate, polyoxyethylene 20 sorbitan monostearate, polyoxyethylene 20 sorbitan monooleate.

In any of the above compositions, the composition can further include an agent that neutralizes a pathogen (e.g., surfactants, alcohols, antibacterial agents, and/or antiviral agents).

In any of the above compositions, the composition can further include a mucoadhesive polysaccharide/polymer that possesses antiviral activity. In one particular embodiment, the mucoadhesive polysaccharides/polymer that possesses antiviral activity is pectin.

In any of the above compositions, the composition can further include an alcohol including a tertiary or aromatic hydroxyl group (e.g., phenethyl alcohol, benzyl alcohol, or chlorobutanol).

The invention features a composition including: (i) water; (ii) at least one mucoadhesive polymer/polysaccharide selected from gellan, pectin, and combinations thereof; (iii) at least one surfactant selected from benzalkonium chloride, polyoxyethylene 20 sorbitan monooleate, and combinations thereof; and (iv) phenethyl alcohol.

In particular embodiments of the above compositions, the at least one mucoadhesive polymer/polysaccharide includes gellan at a concentration of between 0.1-10% w/v (e.g., a concentration of 0.5±0.25, 3±2, 5±2.5, or 7.5±2.5% w/v).

In certain embodiments of the above compositions, the at least one mucoadhesive polymer/polysaccharide includes pectin at a concentration of between 0.5-10% w/v (e.g., a concentration of 0.75±0.25, 3±2, 5±2.5, or 7.5±2.5% w/V).

In some embodiments of the above compositions, the at least one surfactant includes polyoxyethylene sorbitan monooleate at a concentration of between 0.01-0.5% w/v (e.g., a concentration of 0.025±0.015, 0.075±0.025, 0.1±0.025, 0.25±0.1, 0.35±0.1, or 0.4±0.1% w/v).

In certain embodiments of the above compositions, the at least one surfactant includes benzalkonium chloride at a concentration of between 0.01-1% w/v (e.g., a concentration of 0.025±0.015, 0.075±0.025, 0.1±0.025, 0.25±0.1, 0.35±0.1, 0.5±0.25, or 0.75±0.25% w/v).

In some embodiments of the above compositions, the composition includes between 0.25-1% w/v (e.g., 0.35±0.1, 0.45±0.1, 0.5±0.25, or 0.75±0.25% w/v) phenethyl alcohol.

In a particular embodiment of the above compositions, the composition includes 0.2% w/v gellan, 0.75% w/v pectin, 0.05% w/v polyoxyethylene 20 sorbitan monooleate (Tween® 80), 0.01% w/v benzalkonium chloride, and 0.25% w/v phenethyl alcohol.

The compositions of the invention can exhibit a residence time of 4-8 hours when applied to the mucus membrane of a nasal cavity.

In certain embodiments of the above compositions, the composition has a viscosity of 0.01-1 Pa·s. In other embodiments of the above compositions, the composition has a viscosity of 0.01-0.1 Pa·s.

In some embodiments of the above compositions, the at least one mucoadhesive polymer/polysaccharide includes a polysaccharide having an average MW in the range of 10,000 to 2,000,000 Da. In other embodiments of the above compositions, the at least one mucoadhesive polymer/polysaccharide includes a polysaccharide having an average MW in the range of 50,000 to 500,000 Da.

In certain embodiments of the above compositions, the formulation contains less than 0.1% (w/w) solid particles.

In some embodiments of the above compositions, the formulation further includes a therapeutic or diagnostic agent (e.g., an analgesic, an anti-inflammatory, an antihistamine, naltrexone, or melatonin).

In certain embodiments of the above compositions, the at least one mucoadhesive polymer/polysaccharide includes gellan at a concentration of between 0.1-0.4% w/v to achieve a sprayable formulation which forms a mucosal coating that reduces virus transport by at least 90% over a period of 4 hours.

In some embodiments of the above compositions, the at least one mucoadhesive polymer/polysaccharide includes pectin at a concentration of between 0.25%-2% w/v to achieve a sprayable formulation with >90% pathogen neutralization. For example, the composition can include 0.75 +0.05% w/v pectin and benzalkonium chloride at a concentration of less than or equal to 0.1% to achieve >99% pathogen neutralization.

In certain embodiments of the above compositions, the at least one surfactant includes polyoxyethylene sorbitan monooleate at a concentration of between 0.01-0.05% w/v, to achieve a formulation which forms a mucosal coating that enhances the capture of respiratory droplets by >3-fold compared to an uncoated surface, increases the nasal residence time. For example, the mucosal coating that enhances the capture of respiratory droplets by >15-fold at 8 hours post administration, compared to a coating that is otherwise identical, but free of surfactants. The mucosal coating can be formulated such that it does not exhibit mucosal or epithelial toxicity.

In some embodiments of the above compositions, the at least one surfactant includes benzalkonium chloride at a concentration of between 0.005-0.02% w/v, to achieve a formulation which forms a mucosal coating that achieves >90% pathogen neutralization and does not exhibit mucosal or epithelial toxicity. For example, the composition can include 0.010±0.005% w/v benzalkonium chloride and a gellan concentration of less than or equal to 0.2% w/v to achieve >99% pathogen neutralization. In another embodiment, the composition includes 0.010±0.005% w/v benzalkonium chloride and a pectin at a concentration of less than or equal to 1% w/v to achieve >99% pathogen neutralization.

The invention features a method of reducing the risk of exposure to an infectious pathogen at a mucus membrane of a subject, the method including topically applying to the mucus membrane of the subject a composition of the invention. The method can include applying the composition to oral cavity, throat, vagina, nasal cavity, anus, or a wound of the subject.

The invention further features a method of reducing the risk of exposure to an infectious pathogen in a subject, the method including topically applying to a skin of the subject a composition of the invention.

In particular embodiments of the above methods, the subject is a mammal (e.g., a human, dog, cat, horse, or farm animal). In some embodiments of the above methods, the formulation further includes a therapeutic or diagnostic agent (e.g., an analgesic, an anti-inflammatory, an antihistamine, naltrexone, or melatonin).

Other features and advantages of the invention will be apparent from the following description of the preferred embodiments thereof, and from the claims.

### BRIEF DESCRIPTION OF THE DRAWINGS

FIG. 1 shows a schematic overview of Pathogen Capture and Neutralizing Spray (PCANS) functional features. PCANS is a “drug-free” formulation comprising a diverse class of compounds, including biopolymers, surfactants, and alcohols that are listed in the inactive ingredient database (IID) or generally recognized as safe (GRAS) list of the Food and Drug Administration (FDA), and are present as excipients in commercially available nasal/topical formulations. An aqueous solution of PCANS administered using a pocket-sized nasal spray device undergoes a phase transition to form a hydrogel layer in the nasal epithelium. PCANS, which constitutes mucopolysaccharides, surfactants and alcohols, interacts with the divalent ions in the nasal fluid and entangles with mucin chains, forming a crosslinked layer. PCANS enhances the capture of pathogen-laden respiratory droplets from inspired air by preventing their bounce-off. PCANS achieves this by reducing the interfacial tension of the nasal lining, similar to pulmonary surfactants in alveoli. Second, PCANS forms a physical barrier over nasal mucosa to intercept pathogen invasion/colonization. Last, PCANS consists of “non-drug” agents that rapidly neutralize the captured pathogens.

FIG. 2a-2k is a set of graphs depicting results from a screen of biopolymers to identify their sprayable concentrations and also determine a biopolymer that forms an efficient physical barrier against pathogen entry. Viscosity as a function of shear rate up to 40 s⁻¹ at 25° C. for different concentrations of (a) gellan, (b) pectin, (c) hydroxy propyl methyl cellulose (HPMC), (d) carboxymethylcellulose (CMC), (e) Carbopol and (f) xanthan gum in water. The sprayable viscosity window is shown below the dashed line. (g) Storage modulus (G′) of 0.4% (w/v) gellan, 2% (w/v) pectin, 0.5% (w/v) HPMC, 0.5% (w/v) CMC, 0.2% (w/v) Carbopol, and 0.2% (w/v) xanthan gum, without and with simulated nasal fluid (SNF). Amplitude sweep measurements were performed at 37° C. by varying oscillatory strain between 0.005% to 10% at 1 Hz frequency. **** P<0.0001, *P<0.05. n.s., not significant. (h) Amount of Influenza A virus (IAV) that permeated within 4 h through a simulated nasal fluid (SNF)-coated cell strainer (pore size ˜70 μm) or a cell strainer coated with simulated mucus/SNF mixture or a biopolymer/SNF mixture. Permeation of viral particles was quantified by evaluating the viral titer in the chamber below the strainer using plaque assay performed in MDCK host cells. Results are expressed in plaque-forming units (PFU/mL). ** P<0.01, *P<0.05 compared to mucus/SNF, n.s, not significant. Percentage permeation of a fluorescent dye, rhodamine B isothiocyanate through (i) an SNF-coated cell strainer or a cell strainer coated with simulated mucus/SNF mixture or a biopolymer/SNF mixture. **** P<0.0001 compared to mucus/SNF and (j) an SNF-coated strainer or strainer coated with gellan/SNF at different concentrations of gellan. **** P<0.0001 compared to 0.05% w/v gellan/SNF. (k) Percentage drip length of free brilliant green dye or mucoadhesive polymers mixed with brilliant green dye on porcine mucosal tissue. Drip length from the spray area was measured as the distance traversed in 4 h by the biopolymer or free dye from the point of deposition. The percentage drip length of each biopolymer was calculated with respect to the drip length of the free dye. **** P<0.0001 compared to free dye. For g and h, P-values were determined using two-way ANOVA with Tukey's multiple comparisons tests. For i-k, P-values were determined using one-way ANOVA with Tukey's post hoc analysis. Data in a-f are from a single experiment (experiment repeated three times). Data in g-k are means±SD of technical repeats (n=3, each experiment performed at least twice).

FIG. 3a-3c shows the unaltered in-situ gelation and mucoadhesive characteristics of gellan in combination with pectin. (A) Storage modulus of 0.2% w/v gellan, 0.75% w/v pectin and their combination without and with simulated nasal fluid (SNF). (B) Percentage drip length of mucoadhesive polymers on porcine mucosal tissue. (C) Photographic images depicting the drip length of different mucoadhesive polymers or their combination mixed with dye sprayed on the mucosa. Drip length was measured from the distance travelled by the dyed formulation from target area shown in red circle.

FIG. 4 shows the physical barrier property was evaluated by coating the mucoadhesive polymers on a cell strainer for the permeation of influenza A virus or free dye. The amount of virus permeated, or dye diffused (fluorescence intensity) was quantified from the acceptor compartment after 4 hours.

FIG. 5a-5d shows the concentration-dependent effect of gellan on viscosity and spray characteristics. a) Viscosity measured as a function of shear rate up to 40 s⁻¹ at 25° C. for different concentrations of gellan. Quantitative measure of b) plume angle and c) spray coverage area conducted to identify the concentration of gellan with maximum coverage area d) Representative images of plume angle and spray pattern.

FIG. 6a-6u is a set of graphs and schematics showing the results from screenings of mucopolysaccharides, surfactants, and alcohols from the IID and GRAS inventory to identify effective neutralizing agents against respiratory pathogens. a) Table summarizes different components and their concentrations to determine the neutralization ability against respiratory pathogens. Each component was individually evaluated for its viral neutralization potential. IAV and SARS-CoV-2 viral loads in the host cells after 10 or 60 min incubation of the virus with (b, g) different biopolymers, (c, h) different surfactants, and (d, i) different alcohols. Viable viral titer was quantified using plaque assay in MDCK host cells for IAV and focus-forming assay in Vero E6 cells for SARS-CoV-2 virus. Results are expressed in plaque-forming units (PFU/mL) or focus-forming units (FFU/mL). **** P<0.0001, *** P<0.001, ** P<0.01, *P<0.05 compared to 10 minutes of incubation with PBS. n.s, not significant. Viral loads in the host cells after 10 min incubation of (e) IAV and (j) SARS-CoV-2 with different concentrations of pectin and BKC, respectively. ** P<0.01, *P<0.05 compared to PBS. Viral loads in the host cells after 10 min incubation of (f) IAV and (k) SARS-CoV-2 with pectin (0.75% w/v)+polyethylenimine and BKC (0.01% w/v)+bovine serum albumin, respectively. ** P<0.01 compared to PBS. n.s, not significant. (l) Pectin (yellow) binds to the receptor binding site of IAV (purple) at the distal part of hemagglutinin monomer (colored in purple) through hydrophobic interactions with Ser227 and Glu190, and hydrogen bonding with Ser228, Ser186, and Thr187. Blue and red dots in hydrogen bonding maps represent carbon and oxygen atoms, respectively. (m) Chemical interaction of BKC (green) with ACE2 binding motif (red) in the spike protein of SARS-CoV-2. Interaction map reveals the hydrogen bonding of BKC with Tyr505 and Gly496. (n) Aromatic pi-pi interaction of BKC (green) with Phe23 (purple) in the transmembrane domain and with Thr11 (brown) membrane helices. Interaction analysis shows 10 hydrophobic bonds with Phe23 and 8 hydrophobic bonds with Phe26. (0) Viral load in the host cells after 10 min incubation of IAV with pectin (0.75% w/v) in the presence of different concentrations of BKC. **** P<0.0001 compared to PBS. Viral load in the host cells after 10 min incubation of SARS-CoV-2 with BKC (0.01% w/v) in the presence of different concentrations of (p) gellan and (q) pectin. **** P<0.0001 compared to PBS. Effect of surfactants (r, t) and alcohols (s, u) against gram-negative bacteria E. coli and K. pneumoniae using colony-forming unit (CFU) plate count method after 30 and 60 minutes of exposure. Viable bacterial colonies are expressed in CFU/mL. * P<0.05 compared to PBS. For b-d, P values were determined using two-way ANOVA with Tukey's post hoc analysis. For e, f, j, k, o-q, P values were determined using one-way ANOVA. Data in b-k and o-u are presented as Means±S.D of technical repeats (n=3, each experiment performed at least twice). Ser, serine; Thr, threonine; Glu, glutamic acid; Phe, phenylalanine; Tyr, tyrosine; Gly, glycine.

FIG. 7 shows the interaction analysis of sialic acid with receptor binding domain of influenza A virus. Sialic acid (colored in yellow) binds to receptor binding site of hemagglutinin monomer (colored in violet) through hydrophobic interactions with Ser227 and Glu190, and hydrogen bonding with Ser288, Ser186, and Thr187.

FIG. 8a-8k is a set of schematics, graphs, and photomicrographs showing the enhanced capture of respiratory droplet-mimicking aerosols and prolonged residence time of PCANS in the nasal cavity. (a) Experimental design for measuring the capture of respiratory droplet-mimicking aerosol. A twin impinger was used to simulate the aerodynamics of the human respiratory tract. Mucus or gellan (0.02% w/v) and pectin (0.75% w/v) solution (G+P), without or with different concentrations of Tween-80, Tween-20 or BKC was coated on the inner surface of the throat region of the impinger using a nasal spray device. Droplets with mass medial aerodynamic diameter >5 μm and laden with rhodamine B-loaded liposomes (size ˜400 nm) were generated using a jet nebulizer and administered into the impinger under vacuum (15 L/min). Droplet capture was determined by quantifying the fluorescence intensity of rhodamine B in the biopolymer/surfactant mixture or mucus layer. (b) Fold increase in fluorescence intensity with respect to mucus. **** P<0.0001, *P<0.05 compared to mucus. (c) Transepithelial electrical resistance (TEER) across the human nasal epithelial cell (RPMI-2650)-based monolayer at different time points after treatment with only medium or medium containing Triton-X (0.1% w/v) or different concentrations of Tween-80. Surfactant-containing medium was replaced at 4 h with fresh medium to examine impedance recovery. **** P<0.0001, *** P<0.001 compared to untreated control. n.s, not significant. (d) Experimental design for measuring the capture of respiratory droplet-mimicking aerosol using a 3D printed human nasal cavity model (Koken cast). The inner surface of the nasal cavity was coated with mucus, G+P solution or PCANS (the final formulation) using a nasal spray device. The throat part of the model was coupled to a vacuum pump for simulating the respiratory airflow (15 L/min). Nostrils were then exposed to nebulized rhodamine B-loaded liposomes for 1 minute. Droplet capture was determined by quantifying the fluorescence intensity of rhodamine B in the nasal cavity. (e) Fold increase in fluorescence intensity with respect to mucus. ** P<0.01 compared to mucus. n.s, not significant. (f) Experimental outline for the evaluation of the nasal residence time of PCANS in mice. C57BI/6 mice were intranasally administered with 10 μL of free DiR or DiR-loaded PCANS (PCANS/DIR) into each nostril. Mice were euthanized at different time points over 24 h, and nasal cavity was harvested and imaged using an in vivo imaging system (IVIS). (g) Representative images of the nasal cavity excised at different time points. (h) Quantification of fluorescence intensity in the nasal cavity at different time points. (i) Fold change in total flux at 8 h in the nasal cavity relative to G+P. * P<0.05, compared to G+P. n.s, not significant. (i) Experimental design to assess the biocompatibility of PCANS in mouse nasal cavity. 10 μl PCANS or PBS was administered into each nostril of C57BI/6 mice once daily for 14 consecutive days. Animals were euthanized on day 15 and nasal cavity was analyzed histologically. (j) Representative images of H&E-stained sections of nasal turbinate from mice captured using a 4× objective. Insets represent healthy olfactory epithelium (i) and (iii), and lamina propria (ii) and (iv) captured at 20× objective. For b, e and i, P values were determined by one-way ANOVA using Tukey's post hoc analysis. For b, concentrations for each surfactant were compared individually. For c, P values were determined by two-way ANOVA with Tukey's multiple comparison test. Data in b,c, and e are presented as Means±S.D of biological repeats (n=3, each experiment performed at least twice). Data in h and i are presented as Means±SEM (n=5 mice/group).

FIG. 9a-9b shows the influence of tween-80 concentrations on percentage cell viability after 24 and 48 h in the human nasal epithelium (RPMI-2650).

FIG. 10a-10b shows the in vitro release kinetics of (A) tween-80 and (B) IgG and lysophosphatidylcholine (LPC) from the nasal formulation constituted of 0.2% w/v gellan and 0.75% w/v pectin.

FIG. 11a-11b shows the nasal residence of formulation loaded with NIR dye (A) Concentration dependent influence of tween-80 on the residence time of nasal formulation constituted of gellan and pectin after 8 hours of administration. (B) Screening of non-ionic (Span-20) and cationic (BKC) surfactants on the retention of the formulation after 8 hours. Total flux measured from the nasal cavity.

FIG. 12 shows In vivo imaging showing the biodistribution of DiR-loaded PCANS. Left panel shows localization of formulation in the nasal cavity at 2 h and the right panel shows whole body clearance of PCANS at 24 h. Lu-lungs, H-heart, St-stomach, Li-liver, Sp-spleen and K-kidney.

FIG. 13a-13s is a set of graphs and schematics showing that multifunctional PCANS exhibits barrier property, broad-spectrum pathogen neutralization, and long-lasting shelf stability. (a-b) Amount of different viruses that permeated within 4 h through a simulated nasal fluid (SNF)-coated cell strainer or a cell strainer coated with simulated mucus/SNF mixture or PCANS/SNF mixture. Virus permeation was quantified by plaque assay in MDCK cells (IAV), Vero E6 cells (SARS-CoV-2), and Hep-2 cells (RSV and adenovirus). **** P<0.0001, *** P<0.001, ** P<0.01, *P<0.05. n.s, not significant. Amount of (e) E. coli and (f) K. pneumoniae that permeated within 4 h through an SNF-coated cell strainer or a cell strainer coated with simulated mucus/SNF mixture or PCANS/SNF mixture. Bacterial permeation was quantified using a CFU plate count method. **** P<0.0001, *** P<0.001, ** P<0.01. n.s, not significant. (g-k) Viral titer for IAV, SARS-CoV-2, adenovirus, and RSV after treatment with PBS or PCANS. IAV and SARS-CoV-2 were incubated with PCANS for 10 min, while adenovirus and RSV were treated for 30 min. Anti-bacterial activity of PCANS against (k) E. coli and (l) Klebsiella pneumoniae using CFU plate count method after 30 and 10 min incubation, respectively. *** P<0.001, ** P<0.01, *P<0.05. (m) Spray characteristics of PCANS. The droplet size distribution of PCANS was analyzed using a laser diffraction system. Representative images of single time delay plume angle and ovality ratio, captured using a high-speed digital camera and laser light sheet. (n) Experimental design to assess the stability of PCANS in accelerated temperature conditions (40° C.). PCANS was stored in glass amber bottles. Aliquots were taken at different time points to investigate spray characteristics and pathogen neutralization efficacy. (o) Plume angle, (p) ovality, (q) mean droplet diameter and (r) spray deposition area over a period of 60 days. *** P<0.001, ** P<0.01 compared to day 0, n.s, not significant. (s) Percent reduction in the viral load of IAV and SARS-CoV-2 in their respective host cells after 10 min incubation with PCANS aliquoted at different time points in the stability study. * P<0.05 compared to day 0. n.s, not significant. For a-f and o-s, P values were determined using one-way ANOVA with Tukey's post-hoc analysis. For g-l, P values were determined using a two-tailed t-test. Data are presented as Mean±SD of biological repeats (n=3, each experiment performed at least twice).

FIG. 14a-14p shows prophylactic intranasal administration of PCANS prevents virus infection in murine models. (a) Experimental outline for the prophylactic efficacy study. C57BI/6 mice received a single dose (10 μl) of PCANS or PBS before 15 minutes of intranasal inoculation with 250 PFU Influenza A/PR/8/34. One cohort of animals was followed for body weight changes and survival for a period of 10 days. Animals from a second cohort were euthanized on day 2 or 4 after infection to enumerate lung viral titer, inflammatory cell count in bronchoalveolar lavage (BAL) fluid, and inflammatory cytokine levels in lung homogenate. Hematoxylin and eosin (H&E) stained lung tissue sections from animals euthanized were assessed for inflammation. (b) Survival and (c) body weight change of mice over a period of 10 days post-infection. P=0.0007 compared to the PBS-treated group for Kaplan-Meier survival curve. * P<0.01 compared to PBS-treated group for body weight change curves. (d) Viral titer from lung homogenate of mice and (e) percentage reduction in viral load in the lungs on day 2 and 4 post-infection, as quantified by plaque assay performed in MDCK cells. ** P=0.001. (f-i) Inflammatory cell count in BAL on day 2 and 4 after infection. **** P<0.0001, ** P<0.01. Levels of (j) IL-6, (k) TNF-α and (l) IL-1β in lung tissues. **** P<0.0001, ** P=0.005. n.s, non-significant, n.d, not detected. (m) Representative images of H&E-stained lung tissue sections of virus-challenged mice that were prophylactically treated with PBS or PCANS. Histology images were captured using 10× and 40× objectives. Scale bar: 100 μm. High-magnified insets depict the difference in the extent of inflammatory infiltrates. Scale bar: 20 μm. (n) Experimental outline to evaluate time-dependent nasal protection by PCANS. Mice received a single dose of PCANS at 2 or 4 h prior to intranasal inoculation with 100 PFU influenza A/PR/8/34. Animals were euthanized on day 2 post-infection to enumerate lung viral titer (o) Viral titer quantified from lung homogenate and (p) percentage reduction in viral load in the lungs on day 2 post-infection for animals challenged after 2 or 4 h after prophylactic treatment. ** P<0.01, *P<0.05, n.s., not significant. For b, P values were determined using the Gehan-Breslow-Wilcoxon test. For c, P values were determined using one-way ANOVA with Brown-Forsythe. For d and f-l, P values were determined using two-way ANOVA with Tukey's post hoc analysis. For o, P values were determined using one-way ANOVA with Tukey's post hoc analysis. Data in c are presented as Means±SEM (n=6 mice/group). Data in d-l are presented as Means±SEM (n=4 mice/group). Data in o-p are presented as Means±SEM (n=6 mice/group).

FIG. 15 shows the bacteriostatic activity of the formulation constituted of 0.2% w/v gellan and 0.75% w/v pectin with preservatives, phenylethyl alcohol and benzalkonium chloride.

### DEFINITIONS

As used herein, the term “increases the capture of nebulized particles” or “increases the capture of respiratory droplets” refers to the ability of coating compositions of the invention to capture nebulized particles in an ex vivo (e.g., in vitro) model using a twin impinger chamber to simulate the aerodynamics of the human respiratory tract. The inner surface of the chamber is deposited with a coating composition of the invention (e.g., coated with SNF followed by depositing 100 μL of the coating composition) or a control identical in composition except that the control coating contains no surfactant. Fluorescent dye-loaded particles are nebulized (e.g., resulting in droplets with mass median aerodynamic diameter >5 μm). Nebulization is performed at a rate of 30 L/min for 30 seconds. Capture efficiency is assessed by quantifying the fluorescence intensity relative to that observed for the control. The coatings of the invention can increase the capture of nebulized particles by at least 1.5-fold, 2-fold, 3-fold, or 5-fold relative to the control.

As used herein, the term “residence time” refers to the length of time a mucus membrane coated according to the methods of the invention provides a physical barrier that traps particles and droplets. The residence time can be measured using the methods described in Example 4.

As used herein, the term “increases residence time” refers to an increase in the length of time a mucus membrane coated according to the methods of the invention provides a physical barrier that traps particles and droplets. The increase in residence time is measured using the in vivo methods described in Example 4 (e.g., by quantifying the amount of coating material remaining in the nasal cavity, for example at 8 hours post administration). Mice are treated (e.g., intranasally) with a coating composition (e.g., fluorescent dye-loaded) of the invention (e.g., (10 μL/nostril) compared to a control identical in composition except that the control coating contains no surfactant. The coatings of the invention can increase residency time by at least 2-fold, 5-fold, 10-fold, 15-fold, or 30-fold relative to the control. For example, fluorescence signal in the nasal cavity can be quantified at 8 hours.

As used herein, the term “pathogen neutralization” refers to the ability of the compositions and methods of the invention to impede pathogen entry into host cells or impede pathogen growth by either destabilizing the pathogen cell membrane or blocking the receptor-mediated binding/fusion of the pathogen through chemical interactions. Pathogen neutralization for viruses is evaluated as described in Example 2 and 5 by incubating a 50 μL of specific agent or the coating composition of the invention with 50 μL of the virus suspension (10⁴-10⁶ PFU/mL) for 10, 30 or 60 min, followed by 1-min centrifugation and subsequent infection of target cells with the supernatant evaluated using plaque forming or focus-forming assay. Pathogen neutralization for bacteria is evaluated as described in Example 2 and 5 by measuring the bactericidal activity. 50 μL of each agent or the coating composition of the invention is incubated with bacteria (10⁸ CFU/mL) for 30 or 60 min, followed by 1-min centrifugation, and then evaluating the bacterial load in the supernatant using a colony-forming assay.

As used herein, the term “reduces small molecule transport” refers to the ability of coating compositions of the invention to reduce the transport of Rhodamine B isothiocyanate dye (e.g., 0.1 mL) at a concentration of 0.1 mM across (i) a strainer (70 micron pore size) coated with SNF (e.g., 15 μL) plus the coating composition (e.g., 15 μL) of the invention in comparison to (ii) a strainer (70 micron pore size) coated with only SNF (e.g., 15 μL). The dye permeated across the cell strainer is quantified by measuring fluorescence intensity after, e.g., 4 hours. The percent reduction in small molecule transport is based upon relative performance of the composition/SNF-coated and only SNF-coated tests. The coatings of the invention can reduce small molecule transport by at least 90%, 95%, or 99.9% over a period of 4 hours.

As used herein, the term “reduces virus transport” refers to the ability of coating compositions of the invention to reduce the transport of Influenza A virus (e.g., 0.1 mL; 1×10⁵ PFU/mL) (i) a strainer (70 micron pore size) coated with SNF (e.g., 15 μL) plus the coating composition (e.g., 15 μL) of the invention in comparison to (ii) a strainer (70 micron pore size) coated with only SNF (e.g., 15 μL). The amount of virus permeated across the cell strainer is quantified using a plaque assay performed in MDCK host cells, e.g., after 4 hours. The percent reduction in virus transport is based upon relative performance of the coated and uncoated tests. The coatings of the invention can reduce viral transport by at least 90%, 95%, or 99.9% over a period of 4 hours.

As used herein, the term “reducing the risk of exposure to an infectious pathogen” refers to entrapment and/or neutralization of infectious microbes (e.g., bacteria, viruses, and fungi) at a mucus membrane coated with a composition of the invention relative to an uncoated mucus membrane exposed to the same infectious pathogen under the same conditions. The methods of the invention can be used to reduce the number of infectious pathogens that reach the mucus membrane of a subject, and in doing so can reduce the risk of exposure to an infectious pathogen.

As used herein, the term “polysorbate surfactant” refers to a class of nonionic surfactants derived from pegylated sorbitan esterified with fatty acids. Common brand names for Polysorbates include Alkest, Canarcel and Tween. Polysorbate surfactants include, without limitation, polyoxyethylene 20 sorbitan monolaurate (Tween 20), polyoxyethylene (4) sorbitan monolaurate (Tween 21), polyoxyethylene 20 sorbitan monopalmitate (Tween 40), polyoxyethylene 20 sorbitan monostearate (Tween 60), and polyoxyethylene 20 sorbitan monooleate (Tween 80).

As used herein, the term “sorbitan fatty acid ester surfactant” refers to a class of nonionic surfactants derived from sorbitan esterified with fatty acids. Sorbitan fatty acid ester surfactants include, without limitation, sorbitan monolaurate (Span-20 (Atlas/ICI), Crill 1 (Croda), Arlancel 20 (ICI)); sorbitan monopalmitate (Span-40 (Atlas/ICI), Crill 2 (Croda), Nikkol SP-10 (Nikko)), Sorbitan monooleate (Span-80 (Atlas/ICI), Crill 4 (Croda), Crill 50 (Croda)), sorbitan monostearate (Span-60 (Atlas/ICI), Crill 3 (Croda), Nikkol SS-10 (Nikko)); sorbitan trioleate (Span-85 (Atlas/ICI), Crill 45 (Croda), Nikkol SO-30 (Nikko)), sorbitan sesquioleate (Arlacel-C(ICI), Crill 43 (Croda), Nikkol SO-15 (Nikko)), sorbitan tristearate (Span-65 (Atlas/ICI), Crill 35 (Croda), Nikkol SS-30 (Nikko)), sorbitan monoisostearate (Crill 6 (Croda), Nikkol SI-10 (Nikko)), and sorbitan sesquistearate (Nikkol SS-15 (Nikko).

### DETAILED DESCRIPTION

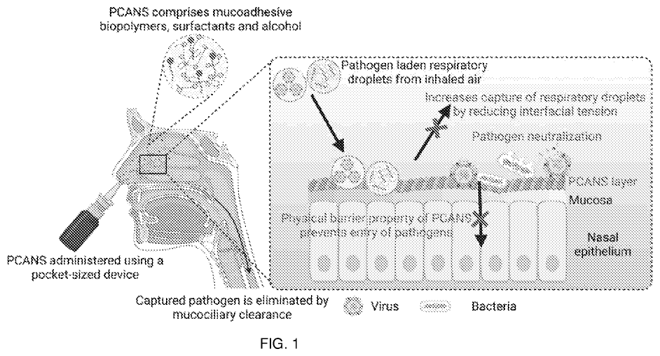

In this study, we describe a nasal spray that integrates unique properties of different classes of compounds such as polysaccharides, surfactants, and alcohols from the inactive ingredient guide (IIG) inventory of the Food and Drug Administration (FDA) for nasal, ophthalmic, and topical routes. We engineered a stable nasal spray formulation, referred to as PCANS, for prophylactic treatment of upper respiratory infections and thereby surpassing the benchmark of conventional nasal sprays against a single airborne pathogen. In addition, we established the minimum gel strength of nasal spray to confer physical barrier protection, which prevents the entry of pathogens to the underlying nasal epithelium (FIG. 1). We adopted a biomimetic approach of capturing foreign particles by reducing the interfacial tension of PCANS as observed with pulmonary surfactants in alveoli. The wettability of PCANS was enriched to promote the interaction of differentially charged viral particles and naturally flush them out through mucociliary clearance, as depicted in FIG. 1. As is described below, we demonstrated long-lasting nasal residence of PCANS and prophylactic protection in animal models. Additionally, PCANS provides a platform with an excellent shelf life and the ability to target relevant airborne pathogens.

The methods and compositions of the invention include polymers and polysaccharides that can act as a physical barrier and/or kill/neutralize pathogens. Following is a non-exhaustive list of polymers and polysaccharides that can act as a physical barrier and/or kill/neutralize pathogens: Cellulose, microcrystalline/carboxymethyl-cellulose sodium/potassium/calcium, hydroxyethyl cellulose, hypromellose, methylcellulose, ethylmethylcellulose, pectin, amidated pectin, xanthan, guar gum, karaya gum, hyaluronic acid, collagen, gelatin, sodium alginate, gellan, kappa carrageenan, lambda carrageenan, iota carrageenan, starch, glucomannan, chitin, chitosan, carboxymethyl chitosan, glycosaminoglycans, dextran, levan, polygalactosamine, amylose, amylopectin, propane-1,2-diol alginate, alginic acid; sodium alginate; potassium alginate; ammonium alginate; calcium alginate; locust bean gum, tragacanth, acacia, ammonium phosphatides, linear polymer of 1-vinyl-2-pyrrolidone and polyethylene glycol, thiolated poly(acrylic acid), poloxamer, cellulose acetophthalate, methyl cellulose, hydroxy ethyl cellulose, poly(amidoamine) dendrimers, poly(dimethyl siloxane) and poly(vinyl pyrrolidone), chitosan-iminothiolane, poly(acrylicacid)-homocysteine, chitosan-thioethylamidine, alginate-cysteine, poly(methacrylic acid)-cysteine, sodium carboxymethylcellulose-cysteine, polyox WSR, carbophil, carbomer, poly(dimethylaminalkyl methacrylates), polyethylene glycol, poly(dimethylaminalkyl acrylates), and the copolymers poly(dimethylaminalkyl methacrylates-co-trimethylaminoalkyl methacryalte) and poly(dimethylaminalkyl acrylates-co-trimethylaminoalkyl acrylate), cationic oligomers or polymer comprising a cationic polysaccharide, cationic copolymer of saccharide and a synthetic cationic monomer, cationic polyalkylene imines, cationic ethoxypolyalkylene imines, cationic poly[N-[3-(dialkylammonio) alkyl] N′ [3-(alkyleneoxyalkylene dialkylammonio) alkyl]urea dichloride], vinyl caprolactam/VP/dialkylaminoalkyl alkylate copolymers, polyquaternium polymers, wherein said cationic oligomer or polymer includes polyquaternium-2, polyquaternium-4, polyquaternium-5, polyquaternium-6, polyquaternium-7, polyquaternium-10, polyquaternium-11, polyquaternium-16, polyquaternium-22, polyquaternium-24, polyquaternium-28, polyquaternium-32, polyquaternium-37, polyquaternium-39, polyquaternium-42, polyquaternium-43, polyquaternium-44, polyquaternium-46, polyquaternium-47, polyquaternium 51, polyquaternium-53, polyquaternium-55, polyquaternium-57, polyquaternium-58, polyquaternium-59, polyquaternium-60, polyquaternium-63, polyquaternium-64, polyquaternium-65, polyquaternium-68, and mixtures thereof.

The methods and compositions of the invention include surfactants. Following is a non-exhaustive list of surfactants that can maximize the capture of respiratory droplets, prolong residence time of the formulation in nasal cavity, and/or kill/neutralize pathogens: polyoxyethylene esters of fatty acids, polyoxyethylene mercaptans and polyoxyethylene alkylamines, polyethoxy ethanol, polyoxyethylene oleyl ether, sorbitan monolaurate, sorbitan monooleate, polysorbate-20, polysorbate-40, polysorbate-60, polysorbate-80 and onyxol, nonoxynol-9, laureth-9, poloxamer-124, octoxynol-9, octyl glucoside, and lauramide DEA, sodium stearoyl-2-lactylate; calcium stearoyl-2-lactylate: stearoyltartrate; sorbitan monostearate; sorbitan tristearate; sorbitan monopalmitate; extract of quillaia; polyglycerol esters of dimerized fatty acids of soya bean oil; oxidatively polymerized soya bean oil, saponins, 1,2-Dipalmitoyl-sn-glycerol-3-phosphoglycerol, sodium salt, dipalmitoylphosphatidylcholine, 1,2-dimyristoyl-sn-glycerol-3-phosphocholine, 1-palmitoyl-2-oleoyl-sn-glycerol-3-phosphocholine, palmitic acid, sodium, potassium, and calcium salts of fatty acids; mono- and di-glycerides of fatty acids; acetic acid esters of mono- and diglycerides of fatty acids; lactic acid esters of mono- and diglycerides of fatty acids: citric acid esters of mono- and diglycerides of fatty acids; tartaric acid esters of mono- and diglycerides of fatty acids; mono- and diacetyl tartaric acid esters of mono- and diglycerides of fatty acids; mixed acetic and tartaric acid esters of mono- and diglycerides of fatty acids; Sucrose esters of fatty acids; sucroglycerides; polyglycerol esters of fatty acids; polyglycerol esters of polycondensed fatty acids of castor oil; propane-1,2-diol esters of fatty acids, sorbitan monolaurate, benzalkonium chloride, and any other cationic surfactants.

The methods and compositions of the invention can include agents that can kill/neutralize pathogens. Following is a non-exhaustive list of other agents that can kill/neutralize pathogens: phenethyl alcohol, benzyl alcohol, citric acid monohydrate, eucalyptol, menthol, methylparaben, phenylmercuric acetate, polyethylene glycol 3350, polyethylene glycol 300, polyethylene glycol3350, polyethylene glycol 400, polyethylene glycol 600, polyethylene glycol 4000, phenol, propylparaben, sodium hydroxide, sorbitol, trisodium citrate dihydrate, propylene glycol, ascorbic acid, aloe, povidone, povidone k12, povidone k15, povidone k30, benzoic acid, crospovidone, polyvinyl alcohol, sodium iodide, lysine acetate, tromethamine, sodium carbonate monohydrate, peppermint oil, saccharin sodium, cyclodextrin, amylmetacresol, chlorohexagluconate, polyhexamethylene bi-guanide, 2,4-dichlorobenzyl alcohol, hexylresorcinol; and metals, including but not limited to manganese (Mn), mercury (Hg), silver (Ag), zinc (Zn), tin (Sn), iron (Fe), copper (Cu), aluminum (Al), nickel (Ni), and cobalt (Co). Nonlimiting examples of a metal compound suitable for use herein include the metal compounds referred to as salicylates, fumarates, benzoates, glutarates, lactates, citrates, malonates, acetates, glycolates, thiosalicylates, adipates, succinates, gluconates, aspartates, glycinates, tartarates, malates, maleates, ascorbates, chlorides, sulphates, nitrates, phosphates, fluorides, iodides, pidolates, and mixtures thereof. The acetates, ascorbates, chlorides, benzoates, citrates, gluconates, glutarates, lactates, malates, malonates, salicylates, succinates, sulphates, and mixtures thereof are preferred metal compounds.

The methods and compositions of the invention can include therapeutic agents that be encapsulated in the formulation for drug delivery. Following is a non-exhaustive list of therapeutic agents that be encapsulated in the formulation for drug delivery: antibiotics, such as streptomycin and vanamycin; antiviral and antiretroviral agents, such as tenofovir, emtricitabine and ribarivin; antibacterials, such as tetracycline, quinolines and kanamycin; antiparasitic agents, such as quinacrine and chloroquine; antihistamines such as cetirizine, chlorpheniramine and diphenhydramine; hormones, such as insulin, progesterone, steroids including corticosteroids, estrogens; nonsteroidal anti-inflammatory analgesics, such as salicylic acid, ibuprofen, naproxen; opioid-type analgesics, such as morphine, oxycodone, fentanyl, codeine and hydromorphone; narcotic antagonists, such as naltrexone, naloxone, and buprenorphine; anesthetics, such as lidocaine and bupivacaine; anti-psychotics, such as risperidone and olanzapine; anticholinergics, such as atropine, tiotropium, and ipratropium; B-adrenergic agents, such as salbutamol, salmeterol and formoterol; cardioactive agents, such as nitroglycerin, papaverine and digitalis; vaccines against diseases such as hepatitis, mumps-measles-rubella, influenza, COVID-19, MERS and polio; cell-proliferation agents, such as fibronectin, epidermal growth factor, interleukin-1 and human growth hormone; anti-cell-proliferation agents and chemotherapeutics, such as cisplatin, 5-fluorouracil, paclitaxel, doxorubicin, docetaxel, tumour necrosis factor, fibroblast growth factor antagonists, and various monoclonal antibodies; as well as hypnotics, sedatives, immunosuppressives, mucolytics, decongestants such as phenylephrine, oxymetazoline, naphazoline, ephedrine, pseudoephedrine, propylhexedrine and phenylpropanolamine, anticonvulsants, antidepressants, antispasmodics, antipruritics, vasodilators, humectants, herbal medications, aloe, essential oils, antidiuretics, antihypertensives, sleep promoting agents, melatonin, benzodiazepines, midazolam, diazepam and diamorphine, neutraceuticals, naphazoline hydrochloride, menthol, cannabinoids, vitamins, drug used for substance used disorder, benzodiaepines, barbiturates, disulfiram, naltrexone, acamprosate, clonidine, nicotine, bupropion, buprenorphine, contraceptives, cholesterol-lowering drugs, nasal hydration promoting agents, nutritional agents, xylometazoline, fluticasone, and other biological compounds such as DNA and RNA nucleic acid sequences and oligonucleotides, peptides, lipids, polysaccharides, proteins, enzymes, antibodies or ribozymes, interferons, Toll like receptor agonist such as nucleoside analogues, imidazoquinoline and its derivatives, and retinoic acid and its derivatives, and combinations thereof.

### EXAMPLES

### Results

### Example 1: Leveraging Biopolymers to Restrict Pathogen Entry with a Physical Barrier

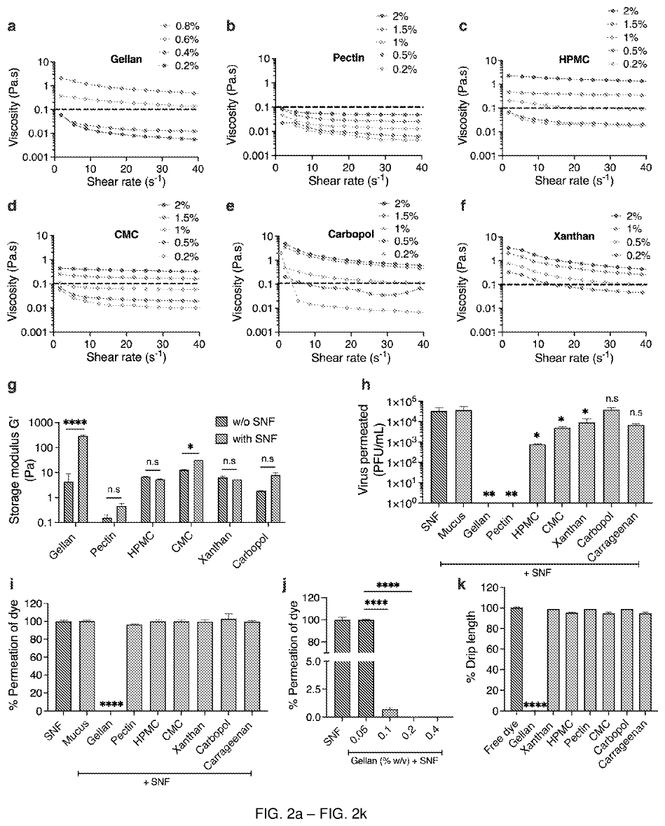

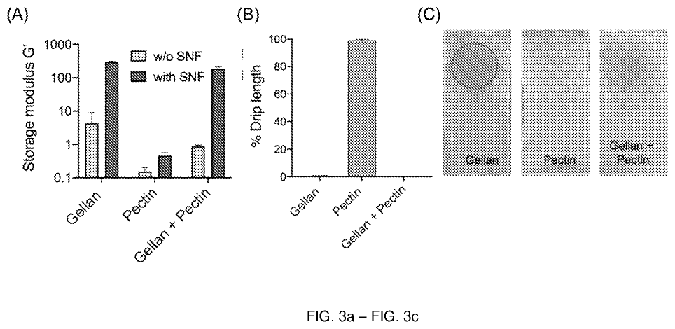

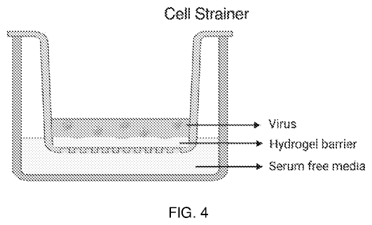

We selected mucoadhesive biopolymers that are listed in the IID or GRAS list of the FDA and are present as excipients in commercially available nasal/topical formulations. Specifically, gellan, pectin, hydroxypropyl methylcellulose (HPMC), carboxymethyl cellulose sodium salt (CMC), carbopol, and xanthan gum were selected. The biopolymers were screened for their ability to impart physical barrier property to PCANS. Since a metered spray device would be used to administer PCANS, we first identified sprayable concentration of each biopolymer by performing rheological measurements (FIG. 2a-f). Dynamic viscosity curves were generated using a rotational rheometer by varying shear rates up to 40 s⁻¹, which is within the lower limits of shear rates encountered while dispensing formulations through a nasal spray device. Concentrations that exhibited a viscosity of less than 0.1 Pa·s were considered ‘sprayable’. Next, we determined the mechanical strength of each biopolymer at the highest sprayable concentration before and after the addition of simulated nasal fluid (SNF). SNF was added to mimic the physiological environment in the nasal cavity. Mechanical strength was measured using a rotational rheometer and quantified as storage modulus (G′), which represents the amount of structure present in a material In the presence of SNF, gellan showed the highest G′ as compared to other biopolymers (FIG. 2g), indicating its superior mechanical strength. Gellan showed a 100-fold increase in its G′ in the presence of SNF (FIG. 2g), which is consistent with its ability to undergo in situ gelation under physiological conditions. Mono and divalent cations present in the SNF complex with glucuronic monomeric units of gellan to form a crosslinked hydrogel. Compared to gellan, other biopolymers showed minimal or no increase in their storage modulus, suggesting poor in situ gelation. We also observed that addition of pectin to gellan didn't compromise it's in situ gelation ability or its storage modulus in the presence of SNF (FIG. 3a). To investigate physical barrier property of biopolymers, a trans-membrane assay was devised (FIG. 4), which involved evaluating the transport of IAV through an SNF-coated cell strainer (pore size ˜70 μm) or a cell strainer coated with simulated mucus/SNF mixture or a biopolymer/SNF mixture. After 4 h, the viral titer in the chamber below the strainer was quantified by performing a plaque assay in Madin-Darby canine kidney (MDCK) host cells. Consistent with its excellent mechanical strength, Gellan/SNF reduced the transport of IAV particles by >4-log fold (99.99%) as compared to only SNF-coated or mucus/SNF-coated strainers (FIG. 2h). Xanthan/SNF, CMC/SNF and HPMC/SNF also significantly reduced the IAV transport, but not as efficiently as gellan/SNF. Interestingly, despite significantly lower mechanical strength of pectin/SNF as compared to gellan/SNF, it intercepted the IAV transport with similar efficiency as gellan/SNF. Carrageenan, a biopolymer used in previously reported and commercially available chemoprophylactic nasal sprays, was used as a control and did not reduce IAV transport in the presence of SNF.

Reduction in the transport of IAV particles by anionic biopolymers could be a result of their physical barrier property and/or electrostatic interactions between their negatively charged polymeric chains and the positively charged capsid of IAV. To decouple the effects of physical barrier property and electrostatic interactions, we studied the transport of a low molecular weight anionic dye, rhodamine B isothiocyanate, that would abate electrostatic interactions with the anionic biopolymers. Gellan/SNF resulted in 100% reduction in the transport of the dye, confirming excellent physical barrier property (FIG. 2i). Other biopolymers did not reduce the transport of the dye, indicating their poor physical barrier property. This also indicates that the reduction in IAV transport by pectin was primarily mediated via the electrostatic interaction of pectin's chains with the virus capsid. Interestingly, gellan/SNF reduced the transport of rhodamine B dye in a concentration-dependent manner (FIG. 2j). A 0.2% w/v concentration of gellan also reduced the transport of E. coli bacteria by >8-log fold (100%), suggesting it's broad spectrum physical barrier property to limit the transport of both viruses and bacteria. To conclude, gellan at a concentration of 0.2% w/v and above impeded the transport of rhodamine B dye, E. coli, and IAV by 100%.

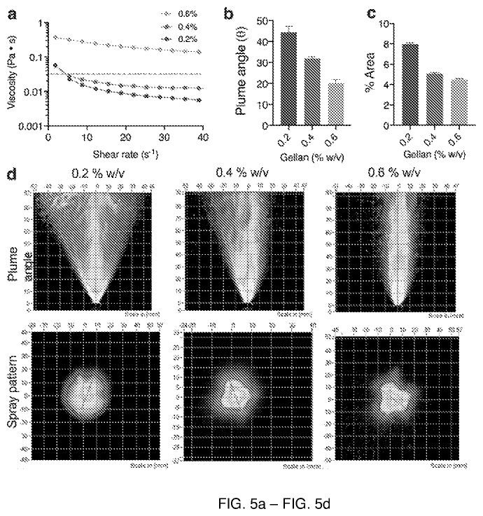

To ensure maximum coverage of the nasal cavity, we evaluated the spray characteristics of gellan at a concentration of 0.2% w/v or higher with a hydraulic spray nozzle. Plume geometry and spray coverage were measured with a high-speed image acquisition system. Increasing gellan concentration resulted in a significant reduction in the angle of emitted plume of the spray (defined as ‘plume angle’) and coverage area (FIG. 5). Henceforth, we used 0.2% w/v gellan due to its superior physical barrier property, plume angle, and coverage area, as compared to other concentrations.

Next, we evaluated the retention ability of gellan and other biopolymers at the mucosal tissue upon spraying. Mucosal retention was measured as the drip length, defined as the distance traversed in 4 h by the biopolymer from the point of deposition on sheep's intestinal mucosa placed vertically. To visualize dripping, biopolymers were mixed with a brilliant green dye. The percentage drip length of each biopolymer was calculated with respect to the drip length of the free dye. Gellan (0.2% w/v) demonstrated excellent mucosal retention with zero drip length (FIG. 2k and FIGS. 3b and c). Other biopolymers, including carrageenan, which was used as a control showed >95% drip length, indicating poor mucosal retention. Gellan's superior mucosal retention is attributed to its ability to strongly entangle with mucin glycoprotein in the mucosal tissue during the sol-gel transition.

### Example 2: Identification of Pathogen-Specific Neutralizing Agent

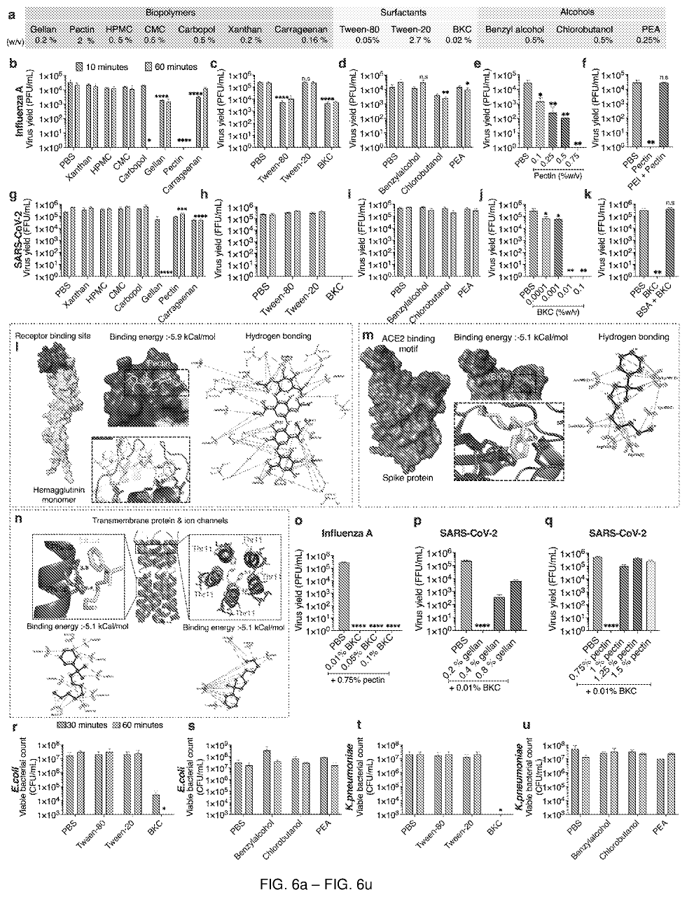

To impart PCANS a broad-spectrum pathogen neutralization ability, we screened agents from three different classes of compounds, including biopolymers, surfactants, and alcohols. These compounds were selected based on their previously reported ability to neutralize different types of pathogens. To maximize safety and translatability of PCANS, we only selected agents that are listed in the IID or GRAS list of the FDA and are present as excipients in commercially available nasal/topical formulations (FIG. 6a). We first evaluated the neutralization ability of these agents against viruses. Neutralization was studied in vitro by incubating each agent individually with either IAV or SARS-CoV-2 for 10 or 60 min, followed by 1-minute centrifugation and subsequent infection of target cells with the supernatant evaluated using plaque forming or focus-forming assay. We chose IAV and SARS-CoV-2 due to their high prevalence worldwide as respiratory viruses and also due to a difference in their capsid proteins and charge. Biopolymers were evaluated at their highest sprayable concentration, except for gellan and carrageenan. Gellan was evaluated at 0.2% w/v due to its superior physical barrier property compared to 0.1% w/v concentration and superior spray pattern compared to 0.4% w/v concentration. Carrageenan, used as a control, was evaluated at 0.16% w/v, as this concentration is present in a commercially available chemoprophylactic nasal spray. Surfactants and alcohols were evaluated at the highest concentration previously used in humans via nasal route. Compared to carrageenan, pectin exhibited superior neutralization of IAV, regardless of the incubation time, and demonstrated a 4-log fold (99.99%) reduction in viral titer in the host cells in comparison to PBS (FIG. 6b). Ten min of incubation with carbopol did not reduce the IAV titer, but a 4-log fold (99.99%) reduction was observed with 60 min of incubation. Gellan exhibited similar neutralization of IAV as carrageenan, resulting in only a 1-log fold (90%) reduction in viral load in the host cells. For SARS-CoV-2, both pectin and carrageenan showed less than a 1-log fold decrease in viral load in the host cells (FIG. 6g). Gellan showed a 4-log fold (99.99%) reduction in the viral titer, but only with 1 h incubation time. Among surfactants, tween 80 and benzalkonium chloride (BKC) showed a 1-log log fold reduction in IAV titer in the host cells, regardless of the incubation time (FIG. 6c). Rapid neutralization of SARS-CoV-2 was observed with BKC, resulting in a 5-log fold (>99.99%) reduction in viral load in the host (FIG. 6h). Alcohols did not neutralize SARS-CoV-2, and minimum neutralization was observed for IAV, resulting in less than 1-log fold (90%) reduction in viral load for chlorobutanol and phenethyl alcohol (PEA) (FIGS. 6d and 6i). Overall, this extensive screening identified pectin and BKC as the most effective agents for rapid neutralization of IAV and SARS-CoV-2, respectively. Neutralization ability of pectin and BKC was found to be dose-dependent (FIGS. 6e and 6j). Minimum concentrations of 0.75% w/v and 0.01% w/v were required for pectin and BKC, respectively, to achieve >4-log fold (>99.99%) reduction in the viral load with 10 min of incubation time.

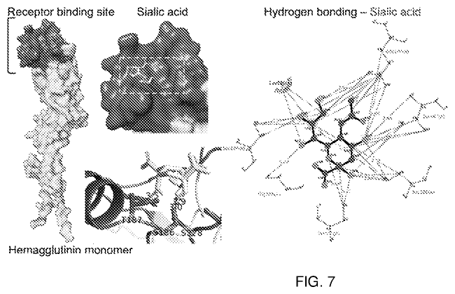

To elucidate the viral neutralization mechanism of pectin and BKC, we performed in silico modeling to determine their binding affinity with the receptor binding domains (RBD) of IAV and SARS-CoV-2, respectively. For IAV, anionic pectin targets RBD at the distal part of hemagglutinin, which is positively charged, thus averting the virus entry into the host cell (FIG. 6l). Compared to the host ligand sialic acid present in mucin, pectin showed stronger binding to RBD through distant hydrogen bonding with Se228, Ser186, and Thr187 and hydrophobic linkage with Ser227 and Glu190 (FIG. 7). BKC was found to exhibit hydrophobic interactions with the ACE2 binding motif of spike protein of SARS-CoV-2 (FIG. 6m). BKC also showed hydrophobic interactions with Phe23 and Phe26 in membrane helices via pi-pi stacking (FIG. 3m), which can distort the helical conformation of adjacent helices, as aromatic stacking of Phe23 and Phe26 is a prerequisite to stabilizing helix-helix interface of the envelope transmembrane protein. BKC fits into the pentameric ion channels at the N terminus of the transmembrane domain through interaction with Thr11 and potentially blocks the influx/efflux of ions (FIG. 6n). To determine the role of electrostatic interaction in pectin- and BKC-mediated neutralization of IAV and SARS-CoV-2, respectively, we performed a neutralization assay by pre-treating pectin and BKC with counter ions to offset the charge. As anticipated, anionic pectin in the presence of positively charged polyethyleneimine lost its neutralization activity and failed to show a significant reduction in the viral load compared to PBS (FIG. 6f). Likewise, the pretreatment of BKC with negatively charged bovine serum albumin diminished the ability of BKC to reduce the SARS-CoV-2 titer in the host cells (FIG. 6k).

Next, we investigated whether ionic interactions between anionic gellan or pectin with cationic BKC, when present together in a formulation, would impact the neutralization ability of pectin or BKC. Notably, the neutralization efficiency of pectin (0.75% w/v) against IAV remained conserved even with a dose-dependent increase in BKC up to a concentration of 0.1% w/v (FIG. 60). Neutralization efficiency of BKC (0.01% w/v) against SARS-CoV-2 was not impacted by gellan or pectin at 0.2% w/v or 0.75% w/v concentrations, respectively, but reduced at higher concentrations (FIG. 6p,q). These results further underscore that the concentration of each agent is critical for efficient neutralization.

Finally, we also screened surfactants and alcohols to assess their neutralization ability against bacteria, including E. coli and Klebsiella pneumoniae (K. pneumoniae). Neutralization was determined by measuring the bactericidal activity. Each agent was individually incubated with either E. coli or K. pneumoniae for 30 or 60 min, followed by 1-minute centrifugation, and then evaluating the bacterial load in the supernatant using a colony-forming assay. BKC was more effective than non-ionic surfactants, resulting in a 4-log fold (99.99%) and 7-log fold (99.99%) reduction in colony-forming units (CFU) of E. coli and Klebsiella pneumoniae, respectively, with an incubation time of 30 min (FIG. 6r, t). Alcohols had a negligible bactericidal effect over the exposure periods of 30 or 60 min (FIG. 6s,u). Altogether, our data on physical barrier property, spray pattern, mucosal retention, and neutralization indicate gellan, pectin, and BKC as the three critical components to formulate PCANS. However, we also incorporated phenethyl alcohol (PEA), as it is commonly added as a stabilizer to nasal formulations to prevent the growth of gram-negative bacteria and fungi and ensure long shelf life.

### Example 3: Surfactants Promote Capture of Respiratory Droplets

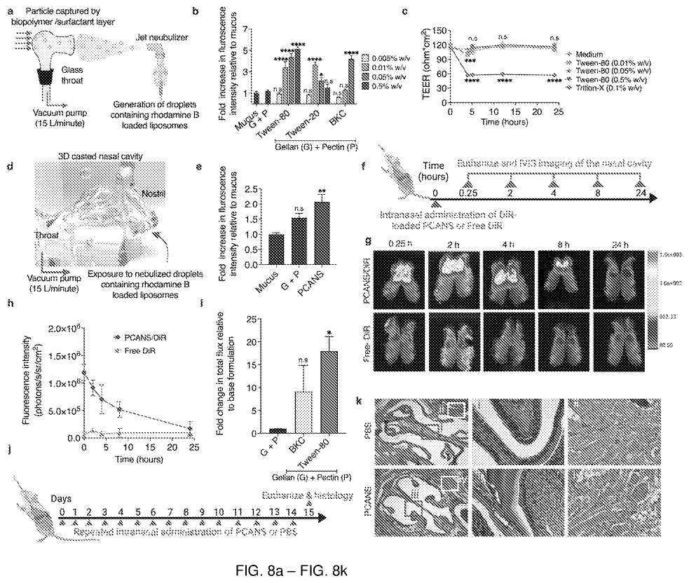

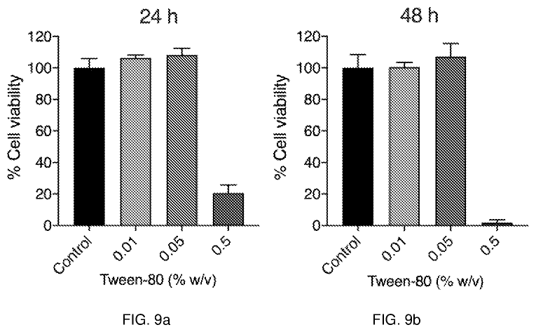

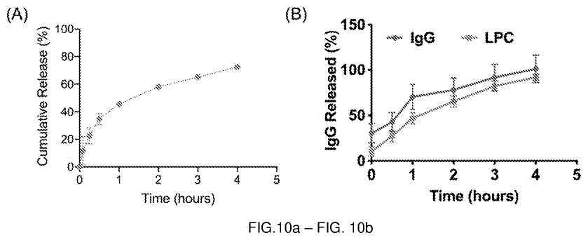

We identified surfactants to reduce interfacial tension of PCANS and reduce the bounce off/escape of respiratory droplets. We evaluated surfactants listed in the IID list, including Tween-20, Tween-80, and BKC. Screening was performed using a twin impinger, which is a glass apparatus that can be used to assess the deposition of aerosolized particles in different regions of the respiratory tract (FIG. 8a). Simulated mucus or a biopolymer mixture of gellan (0.2% w/v) and pectin (0.75% w/v) without or with different concentrations of surfactants was sprayed into the SNF-coated oropharyngeal region of the impinger (FIG. 8a). Droplets with mass medial aerodynamic diameter >5 μm and laden with rhodamine B-loaded liposomes (size ˜400 nm) were generated using a jet nebulizer to mimic pathogen-laden large respiratory droplets. Droplet capture was determined by quantifying the fluorescence intensity of rhodamine B in the biopolymer/surfactant mixture or the mucus layer. Biopolymer mixture without any surfactant showed similar fluorescence intensity as mucus (FIG. 8b). Combining the biopolymer mixture with Tween-80 or Tween-20 at a concentration higher than 0.005% w/v or with BKC at a concentration higher than 0.01% w/v resulted in significant increase in the fluorescence intensity as compared to mucus or only biopolymer mixture, suggesting increased capture of droplets due to surfactants. Compared to Tween-20, BKC and Tween-80 resulted in a significantly higher fold increase in the fluorescence intensity when added to the biopolymer mixture at a concentration of 0.05% w/v or higher (FIG. 8b). At 0.05% w/V concentration, both BKC and Tween-80 containing biopolymer mixtures showed similar fluorescence intensity, which was 4-fold higher than the fluorescence intensity of mucus or biopolymer mixture without a surfactant. Since 0.01% w/v is the most commonly used concentration of BKC in commercially available nasal formulations, and also showed excellent neutralization activity against SARS-CoV-2, we decided to use this concentration in PCANS, even though BKC didn't increase the capture of respiratory droplets at this concentration. To impart respiratory droplet-capturing ability, we decided to proceed with Tween-80 and determined its safe concentration that would not compromise the permeability or metabolic activity of nasal epithelium. To that end, we performed an in vitro assay evaluating the transepithelial electrical resistance (TEER) across the human nasal epithelial cell (RPMI-2650)-based monolayer upon treatment with different concentrations of tween-80. A transient dip of less than 15% in TEER was observed in the monolayer immediately after the addition of Tween-80, irrespective of the concentrations evaluated in this study. However, the TEER reversed rapidly to the original value in less than 1 h after replacing tween-80-containing medium with fresh medium (FIG. 8c). The drop in the TEER for Tween-80 was significantly less compared to Triton-X (negative control), which resulted in a permanent change in the TEER. Second, we evaluated the effect of different concentrations of Tween-80 on the metabolic activity of RPMI-2650 cells upon 24 or 48 h of incubation. Cells incubated with 0.01% or 0.05% w/v tween-80 showed similar metabolic activity as cells incubated in medium. However, Tween-80 (0.5% w/v) resulted in a significant reduction in the metabolic activity of RPMI cells (FIG. 9). Thus, we decided to use 0.05% w/v as the final concentration of Tween-80 in PCANS. We also evaluated the release kinetics of tween-80 from the composite mucoadhesive polymers consisting of 0.2% w/v gellan and 0.75% w/v pectin in presence of SNF. A sustained release of tween-80 was observed from the gel over 4 h (FIG. 10 A). We demonstrated the amenability of the nasal platform to deliver biologics such as immunoglobulin G (IgG) and lipid biomolecule, lysophosphatidylcholine (LPC) over a time course of 4 h (FIG. 10B). Overall, based on our data for physical barrier property, spray pattern, mucosal retention, neutralization, droplet capture, and nasal epithelial cell toxicity, we decided on gellan, pectin, BKC, PEA, and Tween-80 as the final components for PCANS, and validated the respiratory droplet capturing ability of the final formulation using a 3D-printed model of human nasal cavity (Koken cast) with the anatomical intricacies (FIG. 8d). Consistent with the twin impinger results, there was no significant difference in the fluorescence intensity between gellan and pectin mixture, and mucus (FIG. 8e). PCANS, on the other hand, showed a 2-fold higher fluorescence compared to mucus, suggesting the potential of PCANS to increase the capture of pathogen-laden respiratory droplets from inhaled air.

### Example 4: Enhanced Nasal Residence Time of PCANS

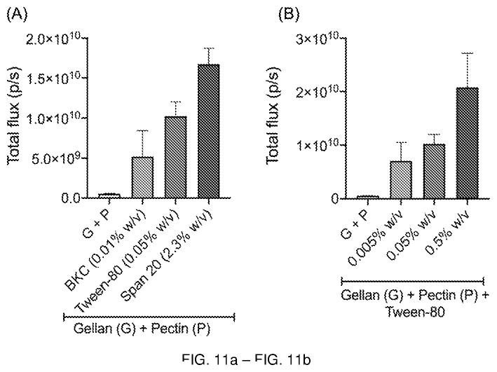

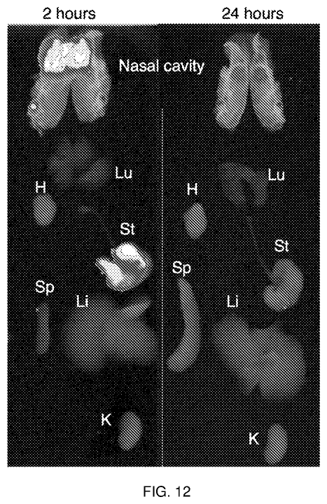

We evaluated the residence time of PCANS in the nasal cavity of mice (FIG. 4f). PCANS (10 μL) mixed with a fluorescent dye-(DiIC18 (7) (1, 1′-Dioctadecyl-3,3,3′,3′-Tetramethylindotricarbocyanine Iodide) (DiR) was administered into both nostrils of C57/BL6 mice. Free DiR was used as a control. Mice were euthanized at different time points over 24 h, and nasal cavity was harvested and imaged using an in vivo imaging system (IVIS) to quantify the fluorescence signal from DIR. Free DiR resulted in negligible fluorescence signal, even at 15 min after administration, suggesting its rapid clearance (FIG. 8g,h). Interestingly, mice administered with DiR-loaded PCANS showed significant fluorescence for up to 8 h, suggesting prolonged nasal residence of PCANS (FIG. 8g,h). We hypothesized that prolonged residence of PCANS is attributed to the presence of surfactants, including Tween-80, BKC and Span-20, which have previously been shown to reduce cilia beat frequency in the nasal cavity. To test our hypothesis, we compared nasal residence time of DiR-loaded mixture of gellan and pectin without or with surfactants. The addition of BKC, Span-20 or tween-80 significantly enhanced the nasal residence of gellan and pectin mixture at 8 h post-nasal administration (FIGS. 8i and 11A). However, tween-80 resulted in significantly higher nasal residence than BKC. Span-20 showed higher nasal residence than tween-80, but also required significantly higher concentration (FIG. 11A). The ability of Tween-80 to enhance the nasal residence of gellan and pectin mixture was found to be concentration-dependent (FIG. 11B). However, considering irreversible nasal epithelial permeabilization and cytotoxicity at 0.5% w/v or higher concentration of Tween-80, we maintained 0.05% w/v in PCANS for further experiments. Notably, nasal administration of DiR-loaded PCANS only showed fluorescence signals in the nasal cavity and stomach, suggesting no systemic absorption (FIG. 12). PCANS was fully cleared at 24 h (FIG. 8g,h, and FIG. 12), resulting in negligible fluorescence signal in both the nasal cavity and the stomach. To confirm safety of PCANS, we performed a repeat-dose toxicity study in healthy mice intranasally administered with PCANS or PBS once daily for 14 consecutive days (FIG. 8j). Hematoxylin and eosin (H&E) stained sections of nasal cavity from both PBS or PCANS-administered mice did not show any inflammation or other gross evidence of toxicity, as evident by a defined lamina propria (FIG. 8k). This connotes the safety of PCANS for daily administration.

### Example 5: Broad-Spectrum Pathogen Neutralization, Barrier Property, and Shelf Stability of PCANS

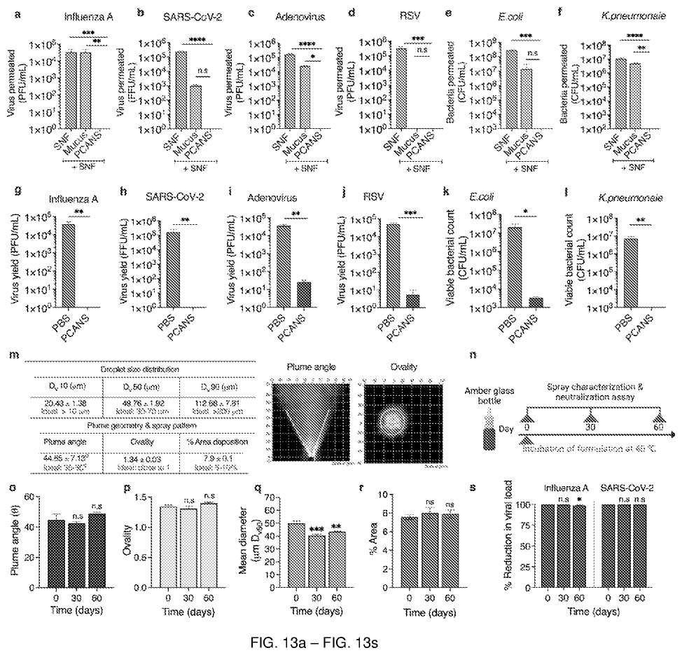

Having identified the final components of PCANS, along with their optimal concentrations, we sought to demonstrate the physical barrier property and neutralization ability of PCANS against a broad spectrum of respiratory pathogens, including enveloped viruses (IAV, SARS-CoV-2, RSV), a non-enveloped virus (adenovirus), and bacteria (E. Coli and K. Pneumonaie). Physical barrier property was evaluated by assessing the transport of pathogens through an SNF-coated cell strainer or a cell strainer coated with simulated mucus/SNF mixture or PCANS/SNF mixture. PCANS/SNF prevented the transport of all the pathogens by >4-log fold (>99.99%) (FIG. 13a-f), suggesting its broad-spectrum physical barrier property. For all pathogens, except RSV, mucus/SNF mixture showed significantly less prevention of pathogen transport compared to PCANS/SNF. PCANS also efficiently neutralized all the tested pathogens within 10 min of incubation time, resulting in >3-log fold (>99.9%) reduction in pathogen load in host cells (FIG. 13g-l). We also evaluated the spray characteristics of PCANS sprayed through a standard and commercially used VP3 multi-dose nasal spray pump (Aptar, USA). The droplet distribution data showed that 10% of PCANS droplets had size >10 μm, and 90% had size <200 μm (FIG. 13m), which is desirable to maximize the deposition in nasal cavity, while minimizing deposition into deep lungs. PCANS resulted in a wide plume angle within the ideal range of 35-55°, an ovality close to 1, covering a circular area of up to 8%, which is in line with the commercial nasal sprays (FIG. 13m).

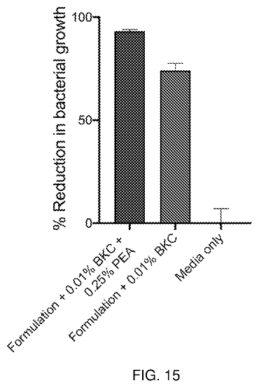

Shelf-stability is a key attribute governing the translational potential of formulations. We tested shelf-stability of PCANS over 60 days at 40° C. temperature, as per the International Conference on Harmonisation (ICH) guidelines for stability testing under accelerated storage conditions (FIG. 13n). Over a period of 60 days, we observed no substantial variations in the spray characteristics, including plume angle, ovality, coverage area, and droplet size distribution (FIG. 13o-r). PCANS also displayed no changes in its neuralization activity over 60 days, resulting in >99.99% reduction in Influenza A and SARS-CoV-2 viral loads in the host cells upon 10 min of incubation (FIG. 13s). Collectively, these data confirm the shelf-stability of PCANS. The bacteriostatic activity of the formulation was evaluated by a microplate assay that employs optical density to quantify bacterial growth. The results showed nearly 100% bacteriostatic activity with the addition of phenylethyl alcohol in the formulation with BKC (FIG. 15). This data emphasizes the significance of phenylethyl alcohol for a long-term stability of the formulation and thwarts the undesired bacterial growth, which stimulates upon the exposure of air and humidity due to intermittent opening of spray bottles.

### Example 6: In Vivo Prophylactic Protection by PCANS Against Influenza

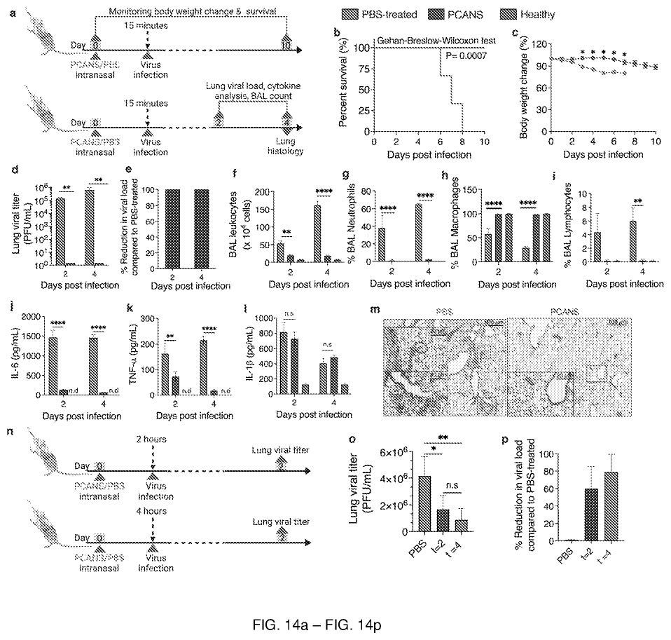

Next, in a proof-of-concept study, we investigated the prophylactic efficacy of PCANS against respiratory infection in vivo. PR8, a mouse-adapted strain of H1N1 Influenza virus, was used to induce infection. PR8 is a highly virulent strain that induces severe respiratory infection in mice, and can be lethal at a dose of 10 PFU. To demonstrate efficacy in vivo, PCANS or PBS (10 μl) was administered prophylactically to both the nostrils of healthy mice on day 0 (FIG. 14a). Fifteen minutes later, animals were challenged intranasally with PR8 (250 PFU), a dose that been previously used by other groups. Remarkably, all mice in the PCANS-treated group survived for at least 10 days after the infection, whereas the PBS-treated group showed 100% lethality by day 8 (FIG. 14b). Over 10 days, no discernible change was observed in the body weight of the PCANS-treated animals, while significant weight loss was observed for PBS-treated ones after 3 days post-infection (FIG. 14c). PCANS also curtailed the lung viral titer to undetectable levels on days 2 and 4 post-infection, resulting in >5-log fold (>99.99%) reduction compared to PBS-treated mice (FIG. 14d-e). Compared to healthy mice, mice infected with PR8 and treated with PBS showed significant differences in the levels of inflammatory cells, including leukocytes, neutrophils, lymphocytes, and macrophages in bronchoalveolar lavage (BAL) fluid (FIG. 14f-i). Prophylactic treatment of mice with PCANS restored the levels of inflammatory cells in BALF to normal. Additionally, cytokine profile from lung homogenate showed a significant reduction of IL-6 and TNF-α levels in PCANS-treated mice, as compared to the PBS-treated group (FIG. 6j-l). No reduction was, however, observed in the levels of IL-1B. Histological examination of lung sections revealed a substantial reduction in leukocyte infiltrates in PCANS-treated mice, as compared to the PBS-treated group, which showed an abundant presence of bronchial and alveolar infiltrates (FIG. 14m). Overall, compared to PBS-treated mice, we observed a significant reduction in pulmonary inflammation score for PCANS-treated group. PCANS also protected mice against the PR8 challenge performed after 2 and 4 h of prophylactic treatment, as evident from significant reduction observed in lung viral titer on day 2, as compared to the PBS-treated group (FIG. 14n-p). Specifically, average reductions of 60% and 78% in lung viral titer were observed for 2 and 4 h challenge groups, respectively; however, there was no statistically significant difference between PCANS-mediated reductions observed in animals challenged after 2 versus 4 h. Overall, our data clearly suggest the potential of nasally administered PCANS to protect against respiratory infection. In conclusion, PCANS presents a promising chemoprophylactic approach against respiratory infections. Besides its potential to act as a first line of defense against respiratory pathogens and emerging variants for which there are no vaccines available, our approach could also be used as an added layer of protection with existing vaccines.

The above-described results were obtained using the following methods.

### Methods

Preparation of biopolymer solutions and PCANS. Biopolymer solutions were prepared by the addition of the biopolymer (0.2 to 2% w/v) to ultrapure deionized sterile water (Invitrogen). The solution was then mixed to attain a homogenous mixture with slight heating at 60° C. Biopolymers including gellan (Gelzan), pectin, carboxymethylcellulose (CMC), hydroxypropyl methylcellulose (HPMC), carrageenan, xanthan gum, and Carbopol were purchased from Sigma Aldrich. To prepare PCANS, 0.4% w/v gellan and 1.5% w/v pectin solutions were mixed in a ratio of 1:1, followed by the addition of tween-80 (Sigma Aldrich) to obtain a final concentration of 0.05% w/v. The solution was then supplemented with benzalkonium chloride (BKC) (Sigma Aldrich) and subjected to immediate mixing by pipetting up and down several times to yield 0.01% w/v in the solution. Finally, 0.25% w/v phenethyl alcohol (Sigma Aldrich) was added, and the pH of the solution was adjusted to 5.5. For cell culture experiments and in vivo efficacy study, the individual components of PCANS were sterile filtered using 0.2 μm PVDF syringe filters (EMD Millipore) and combined as described above.

Preparation of simulated nasal fluid (SNF) and simulated mucus: SNF was prepared by dissolving 1.32 g sodium chloride (150 mM), 447 mg potassium chloride (39.9 mM), and 88.5 mg calcium chloride (5.3 mM) in 150 mL ultrapure deionized sterile water and filtered using 0.2 μm filter. The healthy simulated mucus was formulated by dissolving 0.6 mg mucin from porcine stomach Type II (Sigma Aldrich), 0.8 mg mucin from porcine stomach Type III (Sigma Aldrich), 0.32 mg bovine serum albumin (Sigma Aldrich) in 10 mL ultrapure deionized water containing 20 mM HEPES buffer and 38 mM sodium chloride solution. The mixture was stirred vigorously under slight heating to attain a homogenous solution.

Rheological measurements. Dynamic viscosity behavior of biopolymer solutions was evaluated using a rotational rheometer (Discovery HR-2, TA Instruments) using a 40 mm diameter cone with a geometry angle of 1°. Samples were subjected to a linear shear rate ramp up to 40 s⁻¹ at 25° C. to mimic the strain encountered by the formulation when actuated through the nozzle of the spray device. The viscosity of the biopolymer solution was measured during the upward ramp in triplicates. The sol-gel transition of biopolymer solutions with and without the presence of SNF was evaluated by rotational rheology. The mechanical strength in terms of storage modulus was assessed by applying amplitude sweep with a varying oscillatory strain at 1 Hz at 37° C.

Ex vivo mucosal retention study. Tissue harvested from sheep was cut open to expose the mucosal surface and trimmed down to 75×26 mm. Mucosal tissue was then mounted on a glass slide facing upwards and positioned at 45° to align it with the spray actuation angle. The tissue was initially moistened with SNF using a generic nasal spray device, and excessive fluid was removed with sterile wipes. Brilliant green dye (Sigma Aldrich) loaded polymeric solution was sprayed, keeping the spray nozzle tip at a distance of 5 cm from the slide surface. The slides were examined for runoff/drip after 4 h of spraying. The distance traveled by the polymer solution down the glass slide from the bottom end of formulation deposited on mucosal tissue was measured as drip length. Drip length of free dye was considered 100%.

Cell culture. Madin-Darby canine kidney cells (ATCC®) were cultured in T-175 flasks (CELLTREAT) at 37° C. and 5% CO₂ in DMEM (Gibco) supplemented with 10% fetal bovine serum (FBS) (Gibco) and 1% penicillin-(streptomycin (Invitrogen). Hep2 cells and Vero E6 cells (ATCC®) were cultured at Integrated Biotherapeutics (IBT) Bioservices in T-75 flasks at 37° C. and 5% CO₂ in EMEM supplemented with 10% FBS and 1% penicillin-streptomycin. Human nasal epithelial cells (ATCC) were cultured in T-175 flasks at 37° C. and 5% CO₂ in EMEM supplemented with 10% FBS and 1% penicillin-streptomycin.

Production of NanoLuc Luciferase expressing recombinant SARS-CoV-2. All replication-competent SARS-CoV-2 experiments were performed in a BSL-3 facility at the Boston University National Emerging Infectious Diseases Laboratories. A recombinant SARS-CoV-2 virus expressing a NeonGreen fluorescent protein (rSARS-CoV-2 mNG) 58 was generously provided by the Laboratory of Pei-Yong Shei. To propagate the virus, 1×10⁷ Vero E6 cells were seeded in a T-175 flask one day prior to propagation. The next day, 10 μL of rSARS-CoV-2 mNG virus stock was diluted in 10 mL of OptiMEM, added to cells, and then incubated for 1 h at 37° C. After incubation, 15 mL of DMEM containing 10% FBS and 1% penicillin/streptomycin was added to cells. The next morning, media was removed, cells were washed with 1×PBS and 25 mL of fresh DMEM containing 2% FBS was added. Virus was incubated for an additional 48 h. The supernatant was collected at 72 h, filtered through a 0.22 μm filter, and stored at-80° C. The viral stock was thawed and concentrated by ultracentrifugation (Beckman Coulter Optima L-100k; SW32 Ti rotor) on a 20% sucrose cushion (Sigma-Aldrich, St. Louis, MO) at 25,000×g for 2 h at 4° C. Media and sucrose were then discarded, pellets were dried for 5 min at room temperature, and viral pellets were resuspended in 100 μL of cold 1× PBS at 4° C. overnight. The next day, concentrated virus was combined, aliquoted and stored at −80° C.

In vitro physical barrier assay. A 70 μm pore size mesh cell strainer was coated with 15 μL of mucus, or a biopolymer solution, or PCANS. The formulation was spread evenly using a sterile stainless-steel spatula with a tapered end. To facilitate in situ gelation, 15 μL of SNF was added, covering the entire surface of the strainer. The strainer was placed in a 6-well plate containing 0.9 mL of serum-free DMEM (for virus/bacteria penetration) or ultrapure deionized water (for rhodamine B isothiocyanate penetration) in each well, and 0.1 mL of diluted virus (˜1×10⁵ PFU/mL)/bacteria (1×10⁷ CFU/mL) stock or rhodamine B isothiocyanate (1 mg/mL) was added to the upper compartment of the strainer. After 4 h of incubation at 37° C., medium or deionized water from the bottom reservoir was retrieved, and the viral titer permeated through the hydrogel layer was quantified using plaque assay for IAV performed in MDCK cells, crystal violet staining for RSV performed in Hep-2 cells, immunostaining for adenovirus performed in Vero E6 cells, focus forming assay for SARS-CoV-2 in Vero E6 cells, and colony forming unit (CFU) plate count method for bacteria, as described in the following sections. The permeation of dye through biopolymer solution/mucus was quantified by measuring the fluorescence intensity using a microplate reader.

In vitro neutralization assay with Influenza A. Neutralization activity of different excipients and PCANS was evaluated by plaque assay. MDCK cells were seeded at a density of 2-3 million cells per well in a 6-well plate and then incubated at 37° C. to achieve ˜80-90% confluency one day before infection. On the day of infection, 50 μL of HKx31 Influenza A virus (H3N2, 5×10⁴-1×10⁵ PFU/mL) (BEI Resources) in infection media (serum-free DMEM containing 3 mg/mL TPCK-trypsin) was pre-treated with 50 μL of PCANS, biopolymer solution, surfactant solution, alcohol solution or PBS. Samples were vortexed for 10 seconds and incubated at 37° C. for 10 or 60 min. After incubation, samples were centrifuged for 1 min at 1000 RPM, and the supernatant was subjected to a 10-fold serial dilution until eighth dilution using infection medium. MDCK cells were then exposed to pre-treated virus dilutions for 1 h. After infection, an overlay growth medium containing 2× DMEM with 2% agarose (50:50) was poured onto the top of the cell monolayer and incubated for 72 h. The overlay was removed, and cells were then fixed using 1 mL of 10% formalin and left for 1 h at room temperature, followed by the addition of 1% crystal violet for 5-15 min. Wells were washed with water and left to dry out and PFUs were counted to determine the viral titer.

In vitro neutralization assay with SARS-CoV-2. The day prior to infection experiment, 8×10⁴ Vero E6 cells/well were plated in a 24-well plate. To perform neutralization assay, 50 μL of PCANS, biopolymer solution, surfactant solution, alcohol solution or PBS was mixed with 8×10⁴ PFU of SARS-CoV-2 mNG in 50 μL of infection media (OptiMEM (Gibco) containing 3 mg/mL TPCK-trypsin), vortexed and centrifuged briefly prior to incubation at 37° C. for 10 or 60 min. After incubation, samples were centrifuged for 1 minute at 1000 RPM and serially diluted 10-fold until eighth dilution with infection medium. Of each dilution, 200 μL was then plated into a 24-well plate and incubated for 1 h at 37° C. prior addition of 800 μL of 1.2% Avicel (Dupont). Following a 24 h incubation period at 37° C., Avicel was removed, cells were washed with 1×PBS and fixed for 3 h with 10% neutral buffered formalin. Focal forming units (FFU) per mL were determined by counting NeonGreen expressing foci using an Evos M5000 fluorescent microscope (Thermo Scientific).

In vitro neutralization assay with adenovirus and respiratory syncytial virus. The broad-spectrum neutralization potency of PCANS was evaluated against adenovirus type 5 (ADV-5, ATCC, VR-2554™) and respiratory syncytial virus strain A2 (RSV-A2, ATCC, VR-1540™) using plaque assay. Briefly, the day prior to the infection, 1×10⁵ Vero E6 cells/well or 1.5×10⁵ Hep-2 cells/well were plated in a 24-well plate for ADV-5 and RSV-A2, respectively. On the day of infection, 50 mL of PCANS was mixed with 50 mL of virus (1 ×10⁶ PFU/mL of ADV-5 and 2×10⁶ PFU/mL of RSV-A2) in the infection media and incubated at 37° C. for 30 min. The pre-treated mixture was 10-fold serially diluted in infection media after the incubation. Cells were washed with serum-free media before infection and 200 ml of each dilution was transferred to the cells for a 1 h incubation prior to the addition of a 1 mL overlay medium containing methylcellulose. Following a 72 h incubation, the overlay layer was removed, and cells were fixed using 10% formalin with subsequent immunostaining for Vero E6 cells and crystal violet staining for Hep-2 cells. Plaques were counted using a plaque reader (Bioreader-600-Va).

In vitro neutralization assay with bacteria. The neutralization potency of components and PCANS was studied against gram-negative bacteria including Escherichia coli (E. coli) and K. pneumoniae. An overnight culture of bacteria was prepared in 5 mL tryptic soy broth (TSB, Sigma Aldrich) media. On the day of the experiment, bacteria suspension was adjusted to obtain an OD_{600 nm}=0.2, which corresponds to 10\hat{ }8 CFU/mL. A 50 L of bacterial suspension in TSB media was incubated with 50 μL of PCANS, biopolymer solution, surfactant solution or alcohol solution at 37° C. for 10 or 60 min. After incubation, the sample/bacteria mixture was 10-fold serially diluted in 1×PBS, and 10 mL of each dilution was plated onto pre-poured LB (Luria Broth, HiMedia Laboratories Pvt Ltd) agar plates followed by an incubation of 16-18 h at 37° C., 5% CO₂. The plates were then counted for CFUs.

Bacteriostatic evaluation. An overnight culture of E. coli was prepared by inoculating the colonies grown on agar in tryptic soy broth at 37° C. and appropriately diluted to attain an OD₆₀₀ₙₘ=0.2 ˜ 10⁷ CFU/mL. An equal volume of the formulation constituted of 0.2% w/v gellan, 0.75% w/v pectin, 0.01% w/v BKC and 0.25% w/v phenylethyl alcohol was incubated with E. coli suspension for 24 hours. Absorbance was measured, and percent reduction in bacterial growth was expressed with respect to control without the formulation treatment.

TEER assay and in vitro cytotoxicity of tween-80. RPMI 2650 cells were seeded on the apical part of Transwell inserts (6.5 mm polyester membrane ˜ 0.4 μm pore size, Corning) at a density of 1.5×10⁵ cells/cm² in 0.1 mL EMEM. The basolateral compartment of the insert was filled with 0.6 mL EMEM media supplemented with 10% FBS followed by incubation at 37° C. On day 4, the medium was removed from the top of the inserts, and media volume in the bottom well was reduced to 200 μL. Every 2 days the medium was changed, and TEER was measured. An epithelial volt ohmmeter (World Precision Instrument) was used to measure the impedance. Until the monolayer formed with a constant impedance around day 12, cells were grown with an air-liquid interface. On day 12, TEER was measured prior to the treatment of cells with surfactants. 200 μL of medium containing Triton X-100 (0.1% w/v) or tween-80 at different concentrations was added to the insert. Plate was incubated at 37° C. for 4 h. After incubation, wells were replenished with fresh medium, and TEER was measured after 4, 5, 12, and 24 h. The cytotoxic effect of tween-80 at different concentrations was also studied on RPMI 2650 cells. Briefly, 20,000 cells/well were seeded in a 96-well plate and incubated at 37° C. overnight to achieve 70-80% confluency. Tween-80 (0.01, 0.05, and 0.5% w/v) solution in 0.2 mL EMEM media was added to the wells, followed by an incubation for 24 and 48 h. The metabolic activity of RPMI 2650 cells was measured using an XTT (2,3-bis(2-methoxy-4-nitro-5-sulfophenyl)-2H-tetrazolium-5-carboxanilide) assay kit (ATCC) according to the manufacturer's protocol.

Capture of respiratory droplets. The inner surface of a glass twin impinger's (Copley Scientific) oropharyngeal region (denoted by red arrows in FIG. 4a) was coated with SNF followed by spraying the gellan (0.2% w/v) and pectin (0.75% w/v) mixture without or with different concentrations of tween-20, tween-80 or BKC using a VP3 nasal spray pump (Aptar). Droplets with mass median aerodynamic diameter >5 μm and laden with rhodamine B-loaded liposomes (size ˜400 nm) were generated using a jet nebulizer. Nebulized droplets were administered into the impinger under vacuum at a flow rate of 15 L/min for 3 min. The gel was retrieved, and fluorescence intensity was quantified at an excitation and emission wavelength of 543 and 580 nm. Rhodamine B-loaded liposomes were synthesized using the thin-film hydration method⁵⁹. Briefly, the lipids, DSPE-PEG (2000) amine (Avanti Polar lipids), cholesterol (Sigma) and L-a-phosphatidylcholine, hydrogenated (Soy) (HPC, Avanti Polar lipids) were dissolved in chloroform to prepare a 10 mg/mL lipid stock solution in 1:1:3 molar ratio. A 2 mL of lipid stock solution was added to a round-bottom flask containing 0.8 mL of rhodamine B isothiocyanate from a 1 mg/ml stock. The organic solvent was then evaporated using a rotary evaporator for 5 min to form a thin lipid layer. The lipid film was then hydrated using 10 mL ultrapure water (Invitrogen) and silica glass beads were added to the flask to suspend the lipid in the solution with vigorous shaking using the rotary evaporator at 40° C. for 45 min. The hydrated lipid suspension was sonicated (Probe sonicator) at 30% amplitude for 1 min with a 2 sec pulse on and off condition. The size of liposomes was then analyzed using a Zeta Analyzer (Malvern).

To emulate the capture of pathogen-laden droplets in the human nasal cavity, a 3D printed transparent, silicone human nose model (Koken Co, Ltd) was used. The anterior region of the Koken model was deposited with SNF followed by the gellan (0.2% w/v) and pectin (0.75% w/v) mixture or PCANS with a single actuation using a nasal spray pump (Aptar). Koken model was connected to a vacuum pump at an air flow rate of 15 L/min and rhodamine B-loaded liposomes were then nebulized for 1 min. The model was disassembled to retrieve the formulation and captured dye-loaded droplets after nebulization. The capture of droplets was measured by quantifying the fluorescence intensity at an excitation and emission wavelength of 543 and 580 nm.

Spray characterization. Multi-dose nasal spray vials were filled with water or gellan solution or PCANS. The pump (140 μL) with an insertion depth of 1.8 cm (Aptar) was used to study the spray characteristics including plume geometry, spray plume, and droplet size distribution. Three replicate measurements were performed for each sample. Plume geometry and spray pattern were measured using a Spray-View® measurement system (Proveris Scientific, Hudson, MA) at a distance of 30 mm from the nozzle orifice of the actuator. This acquisition system employs a high-speed digital camera and laser light sheet to capture images. Data were analyzed using an image processing software, Viota®. Actuation parameters including velocity, acceleration and hold time, and settings for camera and laser were kept identical across all the samples. Plume geometry measures the angle of plume ejected from the nozzle orifice. Ovality and plume area were evaluated to quantify the spray pattern of the samples. Ovality is defined as the ratio of maximum to minimum cross-sectional diameter of the spray plume. A uniform circular plume with an ovality close to 1 can be considered an optimal condition for nasal sprays. Droplet size analysis of samples was inspected using a Malvern Spraytec® laser diffraction system. The FDA recommends reporting the measurements of size distribution data at D (v,0.1), D (v,0.5), and D (v,0.9) thresholds which correspond to the size of 10%, 50%, and 90% droplets by volume distribution, respectively. It is suggested to have droplet population with D (v,0.1)>10 mm, D (v,0.5) between 30-70 mm and D (v,0.9)<200 mm. Droplet populations smaller than 10 mm have a propensity to induce a non-targeted deposition at the lungs, and droplets greater than 200 mm tend to drip/run off the nasal cavity.

Shelf-stability study. PCANS (15 mL) was filled in a sterile multi-dose nasal sprays (Aptar) capped with the actuator. The nasal spray vials were stored at an accelerated temperature condition (40° C.). Aliquots were retrieved at different time points and evaluated for neutralization activity against IAV and SARS-CoV-2 using plaque forming and focus forming assays, respectively as described above. Aliquots were collected from three different vials. Similarly, 5 mL aliquots were used to evaluate the spray features including spray pattern, plume geometry, and droplet size distribution.

Mice. Animal experiments were conducted according to ethical guidelines approved by the Institutional Animal Care and Use Committee (IACUC) of Brigham and Women's Hospital. Experiments were conducted in 6-8 weeks-old C57BL/6 mice (Jackson Laboratories, USA). Mice were maintained under pathogen-free conditions and randomly assigned to various experiment groups, irrespective of gender. The group size of animals in experiments was decided based on the minimum number of animals required to attain a statistical significance of P<0.05 among different test groups. For mouse model of influenza infection, experiments were conducted in Biosafety Level 2 according to ethical guidelines approved by the Institutional Animal Care and Use Committee (IACUC) of Brigham and Women's Hospital.

In vivo biodistribution and nasal residence. Nasal residence time of the formulation was determine in mice. Briefly, C57BL/6 mice were administered with 10 μL per nostril of free DiR (Thermofisher) or PCANS mixed with DiR at a final concentration of 10 μg/ml). Mice were euthanized at different time points and nasal cavity was harvested and imaged using IVIS (Bruker's In-Vivo Xtreme optical and x-ray in vivo imaging system) at an excitation and emission wavelength of 680/700 nm. Vital organs such as lung, liver, spleen, kidney, and heart, were also imaged at 2 and 24 h time points. To determine the mechanism of long residence time, animals were intranasally instilled with DiR-mixed gellan (0.02% w/v) and pectin (0.75% w/v) mixture without or with BKC and tween-80. After 8 h, animals were euthanized to harvest and image the nasal cavity using Perkin Elmer IVIS Lumina II and the total flux was expressed in (p/sec/m²/sr).

In vivo prophylactic activity of PCANS. Mice were intranasally instilled with 10 mL PCANS or PBS into each nostril under brief anesthesia using isoflurane. After 15 min, animals were challenged with 250 PFU of PR8 intranasally. One cohort of animals was followed for body weight changes and survival for a period of 10 days. Animals were euthanized when the body weight was reduced to 20%. Animals from a second cohort were euthanized either on day 2 or 4 after infection to enumerate lung viral titer, inflammatory cell count in bronchoalveolar lavage (BAL) fluid, and inflammatory cytokine levels in lung homogenate. BAL fluid was isolated by gently instilling saline solution into bronchioles with a catheter inserted through the trachea. The total cells and immune cell types from the collected BAL fluid were quantified using Diff-quik kit as per manufacturer's protocol. For lung viral titer and cytokine profiling, left lung was homogenized and centrifuged at 2000 g for 10 min at 4° C. to collect the supernatant. The obtained supernatant was further used for downstream assays. Viral titer was enumerated using plaque assay with MDCK cells, as detailed above. Cytokine profiling was performed using respective ELISA kits of IL-6, TNF-α, and IL-1β (BioLegend) according to the manufacturer's protocol. Histopathology of the right lung was determined using hematoxylin and eosin staining. To evaluate the time-dependent protection of PCANS, animals were challenged with 100 PFU of PR-8 via intranasal route after 2 or 4 h of PCANS or PBS treatment and euthanized on day 2 post-infection to quantify lung viral titer using plaque assay.

### Statistics

Statistical analysis and graphing were conducted using Graphpad Prism. A one-way ANOVA with Tukey's post hoc analysis was used to compare multiple groups. Two-way ANOVA with Tukey's multiple comparison tests was used to analyze the data with two variables. To evaluate the efficiency of PCANS, survival plots were generated using the Kaplan-Meier survival curve, and the statistical significance of the results was analyzed using the Gehan-Breslow-Wilcoxon test. P values for the body weight changes were determined using one-way ANOVA with Brown-Forsythe post hoc analysis. A P-value of less than 0.05 was considered statistically significant.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations described herein following, in general, the principles described herein and including such departures from the invention that come within known or customary practice within the art to which the invention pertains and may be applied to the essential features hereinbefore set forth, and follows in the scope of the claims.

Other embodiments are within the claims.

1. A mucosal coating composition comprising:
(i) water;
(ii) at least one mucoadhesive polymer/polysaccharide; and
(iii) at least one surfactant.

2. A mucosal coating composition comprising:
(i) water;
(ii) at least one mucoadhesive polymer/polysaccharide at a concentration between 0.1-20% w/v; and
(iii) at least one surfactant at a concentration between 0.005-5% w/v.

3. The mucosal coating of claim 1 or 2, wherein following application to a surface the coating composition forms a barrier that reduces small molecule transport by at least 90% over a period of 4 hours.

4. The mucosal coating of claim 1 or 2, wherein following application to a surface the coating composition forms a barrier that reduces virus transport by at least 90% over a period of 4 hours.

5. The mucosal coating of any one of claims 1-4, wherein following application to a surface the coating composition increases the capture of nebulized particles by at least 1.5-fold.

6. The mucosal coating of any one of claims 1-5, wherein following application to a surface the coating composition increases the residence time of the coating resulting in at least 2-fold higher amount of coating remaining at 8 hours post-application, compared to a coating otherwise identical in composition, but lacking surfactants.

7. The mucosal coating of any one of claims 1-6, wherein the mucoadhesive polymer/polysaccharide is present at a concentration between 0.25-20% w/v.

8. The mucosal coating of claim 7, wherein the mucoadhesive polymer/polysaccharide is present at a concentration between 0.75-20% w/v.

9. The mucosal coating of any one of claims 1-8, wherein the surfactant is present at a concentration between 0.01-5% w/v.

10. The mucosal coating of claim 9, wherein the surfactant is present at a concentration between 0.05-5% w/v.

11. The mucosal coating of claim 10, wherein the surfactant is present at a concentration between 0.5-5% w/v.

12. A mucosal coating composition comprising:
(i) water;
(ii) at least one mucoadhesive polymer/polysaccharide at a concentration between 0.1-10% w/v; and
(iii) at least one surfactant at a concentration between 0.005-5% w/v,
wherein the composition is a sprayable solution.

13. The mucosal coating of any one of claims 1-12, wherein the at least one mucoadhesive polymer/polysaccharide comprises a carboxyl, hydroxyl, sulfate or acetamido group.

14. The mucosal coating of claim 13, wherein the at least one mucoadhesive polymer/polysaccharide is selected from gellan, pectin, HPMC, CMC, xanthan, chondroitin sulfate, alginic acid, hyaluronic acid, and salts or derivatives thereof.

15. The mucosal coating of claim 14, wherein the at least one mucoadhesive polymer/polysaccharide comprises a polymer of glucuronic acid or galacturonic acid.

16. The mucosal coating of claim 15, wherein the at least one mucoadhesive polymer/polysaccharide is selected from pectin, gellan, HPMC, CMC, xanthan and alginic acid, and salts or derivatives thereof.

17. The mucosal coating of any one of claims 1-16, wherein the at least one surfactant is a (a) hydrophilic and (b) non-ionic or cationic surfactant.

18. The mucosal coating of claim 17, wherein the at least one surfactant is selected from polysorbate surfactants, sorbitan fatty acid ester surfactants, and benzalkonium chloride.

19. The mucosal coating of claim 18, wherein the at least one surfactant is selected from polyoxyethylene 20 sorbitan monolaurate, polyoxyethylene 20 sorbitan monopalmitate, polyoxyethylene 20 sorbitan monostearate, polyoxyethylene 20 sorbitan monooleate.

20. The mucosal coating of any one of claims 1-19 further comprising an agent that neutralizes a pathogen.

21. The mucosal coating of claim 20, wherein the agent is selected from surfactants, alcohols, antibacterial agents, and antiviral agents.

22. The mucosal coating of any one of claims 1-21, comprising a mucoadhesive polysaccharides/polymer that possesses antiviral activity.

23. The mucosal coating of claim 22, wherein the mucoadhesive polysaccharides/polymer that possesses antiviral activity is pectin.

24. The mucosal coating of claim 21, wherein the mucosal coating comprises an alcohol comprising a tertiary or aromatic hydroxyl group.

25. The mucosal coating of claim 24, wherein the alcohol is selected from phenethyl alcohol, benzyl alcohol, and chlorobutanol.

26. A composition comprising:
(i) water;
(ii) at least one mucoadhesive polymer/polysaccharide selected from gellan, pectin, and combinations thereof;
(iii) at least one surfactant selected from benzalkonium chloride, polyoxyethylene 20 sorbitan monooleate, and combinations thereof; and
(iv) phenethyl alcohol.

27. The composition of any one of claims 1-26, wherein the at least one mucoadhesive polymer/polysaccharide comprises gellan at a concentration of between 0.1-10% w/v.

28. The composition of any one of claims 1-27 wherein the at least one mucoadhesive polymer/polysaccharide comprises pectin at a concentration of between 0.5-10% w/v.

29. The composition of any one of claims 1-28, wherein the at least one surfactant comprises polyoxyethylene sorbitan monooleate at a concentration of between 0.01-0.5% w/v.

30. The composition of any one of claims 1-29, wherein the at least one surfactant comprises benzalkonium chloride at a concentration of between 0.01-1% w/v.

31. The composition of any one of claims 1-30, wherein the composition comprises between 0.25-1% w/v phenethyl alcohol.

32. The composition of any one of claims 1-31, wherein the composition comprises 0.2% w/v gellan, 0.75% w/v pectin, 0.05% w/v polyoxyethylene 20 sorbitan monooleate (Tween® 80), 0.01% w/v benzalkonium chloride, and 0.25% w/v phenethyl alcohol.

33. The composition of any one of claims 1-32, wherein the composition exhibits a residence time of 4-8 hours when applied to the mucus membrane of a nasal cavity.

34. The composition of any one of claims 1-33, wherein the composition has a viscosity of 0.01-1 Pa·s.

35. The composition of claim 34, wherein the composition has a viscosity of 0.01-0.1 Pa·s.

36. The composition of any one of claims 1-35, wherein the at least one mucoadhesive polymer/polysaccharide comprises a polysaccharide having an average MW in the range of 10,000 to 2,000,000 Da.

37. The composition of claim 36, wherein the at least one mucoadhesive polymer/polysaccharide comprises a polysaccharide having an average MW in the range of 50,000 to 500,000 Da.

38. A composition of any of the claims 1-37, wherein the formulation contains less than 0.1% (w/w) solid particles.

39. A composition of any of the claims 1-32, wherein the formulation further comprises a therapeutic or diagnostic agent.

40. The composition of claim 39, wherein the formulation comprises an analgesic, an anti-inflammatory, an antihistamine, naltrexone, or melatonin.

41. The composition of any one of claims 1-40, wherein at least one mucoadhesive polymer/polysaccharide comprises gellan at a concentration of between 0.1-0.4% w/v to achieve a sprayable formulation which forms a mucosal coating that reduces virus transport by at least 90% over a period of 4 hours.

42. The composition of any one of claims 1-40, wherein at least one mucoadhesive polymer/polysaccharide comprises pectin at a concentration of between 0.25%-2% w/v to achieve a sprayable formulation with >90% pathogen neutralization.

43. The composition of claim 42, wherein the composition comprises 0.75±0.05% w/v pectin and benzalkonium chloride at a concentration of less than or equal to 0.1% to achieve >99% pathogen neutralization.

44. The composition of any one of claims 1-43, wherein the at least one surfactant comprises polyoxyethylene sorbitan monooleate at a concentration of between 0.01-0.05% w/v, to achieve a formulation which forms a mucosal coating that enhances the capture of respiratory droplets by >3-fold compared to an uncoated surface, increases the nasal residence time.

45. The composition of any one of claims 1-41, wherein the at least one surfactant comprises benzalkonium chloride at a concentration of between 0.005-0.02% w/v, to achieve a formulation which forms a mucosal coating that achieves >90% pathogen neutralization and does not exhibit mucosal or epithelial toxicity.

46. The composition of claim 45, wherein the composition comprises 0.010±0.005% w/v benzalkonium chloride and a gellan concentration of less than or equal to 0.2% w/v to achieve >99% pathogen neutralization.

47. The composition of claim 45, wherein the composition comprises 0.010±0.005% w/v benzalkonium chloride and a pectin at a concentration of less than or equal to 1% w/v to achieve >99% pathogen neutralization.

48. A method of reducing the risk of exposure to an infectious pathogen at a mucus membrane of a subject, said method comprising topically applying to the mucus membrane of the subject a composition of any one of claims 1-47.

49. The method of claim 48, wherein the composition is applied to oral cavity, throat, vagina, nasal cavity, anus, or a wound.

50. A method of reducing the risk of exposure to an infectious pathogen in a subject, said method comprising topically applying to a skin of the subject a composition of any one of claims 1-47.

51. The method of any one of claims 48-50, wherein the subject is a mammal.

52. The method of any one of claims 48-50, wherein the subject is a human, dog, cat, or farm animal.
